SEQUENCE LISTING (13 sequences)

SEQ ID NO: 1 - DNA, 8126 bp, synthetic construct
  misc_feature 1..8126: note = Synthetic Construct
  misc_feature 1..8126: note = MISC_FEATURE - srRNA vector AR86-Girdwood Hybrid 1, without heterologous GOI
  source 1..8126: mol_type = other DNA
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaa

SEQ ID NO: 2 - DNA, 8186 bp, synthetic construct
  misc_feature 1..8186: note = Synthetic Construct
  misc_feature 1..8186: note = MISC_FEATURE - srRNA vector AR86-Girdwood Hybrid 2, without heterologous GOI
  source 1..8186: mol_type = other DNA
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaa

SEQ ID NO: 3 - DNA, 8133 bp, synthetic construct
  misc_feature 1..8133: note = Synthetic Construct
  misc_feature 1..8133: note = MISC_FEATURE - srRNA vector AR86-Girdwood Hybrid 3, without heterologous GOI
  source 1..8133: mol_type = other DNA
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 4 - DNA, 8187 bp, synthetic construct
  misc_feature 1..8187: note = Synthetic Construct
  misc_feature 1..8187: note = MISC_FEATURE - srRNA vector AR86-Girdwood Hybrid 4, without heterologous GOI
  source 1..8187: mol_type = other DNA
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 5 - DNA, 9831 bp, synthetic construct
  misc_feature 1..9831: note = Synthetic Construct
  misc_feature 1..9831: note = MISC_FEATURE - srRNA vector AR86-Girdwood Hybrid 1, with exemplary GOI (HA)
  source 1..9831: mol_type = other DNA
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctatggagaaaatcgtcctcctgtttgctatagtgtcccttgtgaagagcgatcagatctgcatagggtatcatgccaataattccaccgaacaagtggacactattatggaaaagaatgtcacagttacacatgctcaggatatcttggaaaaaaaacacaacggaaagctctgcgatctcgatggtgtaaaaccacttatcctgcgggactgctctgttgcagggtggctgcttggaaaccccatgtgtgacgaatttatcaacgtccccgaatggtcttacatagttgaaaaagcaaatcctgtcaatgacctgtgctaccccggagactttaacgattatgaagagctgaagcatcttcttagtcgaatcaaccattttgagaagatccagattatcccaaagagctcttggagctcacatgaagcaagcctcggggtatcatctgcctgcccctatcaagggaagtctagtttcttcagaaacgtcgtgtggctcatcaaaaagaattcaacttaccctaccatcaagcgaagttataataatacaaatcaagaagatctgctggtgttgtggggcatacatcatcccaatgacgccgccgaacaaacaaagctctaccaaaatcccaccacttacatttctgtggggacatccactctcaaccaaaggctcgtgccacgcatcgctactcggagtaaagtcaatggacagtctgggcgaatggagtttttttggacaattctgaaacctaatgacgccataaacttcgagagcaacgggaacttcatcgcaccagagtatgcctataaaattgtgaaaaaaggcgattccacaattatgaagtctgaattggaatacggaaattgcaataccaaatgccagacaccaatgggtgccataaactcctctatgccctttcataacattcacccacttacaattggcgaatgtccaaaatatgtaaaatcaaatcgcttggttttggctacaggtttgcggaattctcctcaacgagaaagacgccgaaagaagagaggactgttcggtgcaatcgccggtttcatagagggaggttggcaaggaatggtagacggctggtacggttatcaccattcaaatgaacaaggttctgggtatgctgctgataaagaaagcacacaaaaggcaatcgatggcgtaaccaataaagtgaacagcataatcgacaagatgaatacccaatttgaagccgtagggagggaatttaataatctcgaacggcggatcgagaatttgaataagaaaatggaagatggatttttggacgtatggacatataatgccgaattgttggtccttatggagaacgaaagaacactggactttcatgactccaacgtcaagaatttgtatgacaaggtgcgcctccaactccgggataacgctaaagaactcggaaatggttgtttcgagttttaccacaagtgcgataatgagtgcatggaaagcgtgcgaaacggtacttacgattatccccaatattcagaggaagcccgactgaaacgggaggaaatcagtggcgtgaaattggaaagcatcggcatttatcagattcttagcatctatagtactgtcgcatcctctctggccctggctattatggttgctggcctctcactctggatgtgctctaacgggtccctccagtgccggatttgcatatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaa

SEQ ID NO: 6 - DNA, 9885 bp, synthetic construct
  misc_feature 1..9885: note = Synthetic Construct
  misc_feature 1..9885: note = MISC_FEATURE - srRNA vector AR86-Girdwood Hybrid 2, with exemplary GOI (HA)
  source 1..9885: mol_type = other DNA
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctatggagaaaatcgtcctcctgtttgctatagtgtcccttgtgaagagcgatcagatctgcatagggtatcatgccaataattccaccgaacaagtggacactattatggaaaagaatgtcacagttacacatgctcaggatatcttggaaaaaaaacacaacggaaagctctgcgatctcgatggtgtaaaaccacttatcctgcgggactgctctgttgcagggtggctgcttggaaaccccatgtgtgacgaatttatcaacgtccccgaatggtcttacatagttgaaaaagcaaatcctgtcaatgacctgtgctaccccggagactttaacgattatgaagagctgaagcatcttcttagtcgaatcaaccattttgagaagatccagattatcccaaagagctcttggagctcacatgaagcaagcctcggggtatcatctgcctgcccctatcaagggaagtctagtttcttcagaaacgtcgtgtggctcatcaaaaagaattcaacttaccctaccatcaagcgaagttataataatacaaatcaagaagatctgctggtgttgtggggcatacatcatcccaatgacgccgccgaacaaacaaagctctaccaaaatcccaccacttacatttctgtggggacatccactctcaaccaaaggctcgtgccacgcatcgctactcggagtaaagtcaatggacagtctgggcgaatggagtttttttggacaattctgaaacctaatgacgccataaacttcgagagcaacgggaacttcatcgcaccagagtatgcctataaaattgtgaaaaaaggcgattccacaattatgaagtctgaattggaatacggaaattgcaataccaaatgccagacaccaatgggtgccataaactcctctatgccctttcataacattcacccacttacaattggcgaatgtccaaaatatgtaaaatcaaatcgcttggttttggctacaggtttgcggaattctcctcaacgagaaagacgccgaaagaagagaggactgttcggtgcaatcgccggtttcatagagggaggttggcaaggaatggtagacggctggtacggttatcaccattcaaatgaacaaggttctgggtatgctgctgataaagaaagcacacaaaaggcaatcgatggcgtaaccaataaagtgaacagcataatcgacaagatgaatacccaatttgaagccgtagggagggaatttaataatctcgaacggcggatcgagaatttgaataagaaaatggaagatggatttttggacgtatggacatataatgccgaattgttggtccttatggagaacgaaagaacactggactttcatgactccaacgtcaagaatttgtatgacaaggtgcgcctccaactccgggataacgctaaagaactcggaaatggttgtttcgagttttaccacaagtgcgataatgagtgcatggaaagcgtgcgaaacggtacttacgattatccccaatattcagaggaagcccgactgaaacgggaggaaatcagtggcgtgaaattggaaagcatcggcatttatcagattcttagcatctatagtactgtcgcatcctctctggccctggctattatggttgctggcctctcactctggatgtgctctaacgggtccctccagtgccggatttgcatatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaa

SEQ ID NO: 7 - DNA, 9832 bp, synthetic construct
  misc_feature 1..9832: note = Synthetic Construct
  misc_feature 1..9832: note = MISC_FEATURE - srRNA vector AR86-Girdwood Hybrid 3, with exemplary GOI (HA)
  source 1..9832: mol_type = other DNA
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 8 - DNA, 9886 bp, synthetic construct
  misc_feature 1..9886: note = Synthetic Construct
  misc_feature 1..9886: note = MISC_FEATURE - srRNA vector AR86-Girdwood Hybrid 4, with exemplary GOI (HA)
  source 1..9886: mol_type = other DNA
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 9 - DNA, 1704 bp, synthetic construct
  misc_feature 1..1704: note = Synthetic Construct
  misc_feature 1..1704: note = MISC_FEATURE - coding sequence for hemagglutinin (HA) from H5N1
  source 1..1704: mol_type = other DNA
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatt

SEQ ID NO: 10 - DNA, 55 bp, synthetic construct
  misc_feature 1..55: note = Synthetic Construct
  misc_feature 1..55: note = MISC_FEATURE - forward primer
  source 1..55: mol_type = other DNA
gctggagacgtggaggagaaccctggacctatggagaaaatagtgcttctttttg

SEQ ID NO: 11 - DNA, 53 bp, synthetic construct
  misc_feature 1..53: note = Synthetic Construct
  misc_feature 1..53: note = MISC_FEATURE - reverse primer
  source 1..53: mol_type = other DNA
gctggtcgggtcattggggcgtagcggtcaaatgcaaattctgcattgtaacg

SEQ ID NO: 12 - DNA, 18 bp, synthetic construct
  misc_feature 1..18: note = Synthetic Construct
  misc_feature 1..18: note = MISC_FEATURE - bacteriophage T7 RNA polymerase promoter
  source 1..18: mol_type = other DNA
taatacgactcactatag

SEQ ID NO: 13 - DNA, 29 bp, synthetic construct
  misc_feature 1..29: note = Synthetic Construct
  misc_feature 1..29: note = MISC_FEATURE - T7 terminator sequence
  source 1..29: mol_type = other DNA
aacccctctctaaacggaggggttttttt

(19) United States
(12) Patent Application Publication
(10) Pub. No.: US 2024/0400619 A1
(43) Pub. Date: Dec. 5, 2024

(54) MODIFIED ALPHAVIRUSES WITH HETEROLOGOUS NONSTRUCTURAL PROTEINS

(71) Applicant: Replicate Bioscience, Inc., San Diego, CA (US)

(72) Inventors: Nathaniel Stephen WANG, San Diego, CA (US); Shigeki Joseph MIYAKE-STONER, San Diego, CA (US)

(21) Appl. No.: 18/688,697

(22) PCT Filed: Sep. 1, 2022
(86) PCT No.: PCT/US2022/075853

§ 371 Date: Mar. 1, 2024

Related U.S. Application Data
(60) Provisional application No. 63/240,297, filed on Sep. 2, 2021.

(51) Int. Cl. C07K 14/005; A61K 39/00; A61P 31/16; C12N 15/86

(52) U.S. Cl. CPC C07K 14/005; A61P 31/16; C12N 15/86; A61K 2039/55555; A61K 2039/575; C12N 2760/16122; C12N 2760/16134; C12N 2760/20122; C12N 2760/20134; C12N 2770/36122; C12N 2770/36143

(57) ABSTRACT

The present disclosure relates to the field of molecular virology, including nucleic acid molecules comprising modified viral genomes or replicons (e.g., self-replicating RNAs), pharmaceutical compositions containing the same, and the use of such nucleic acid molecules and compositions for production of desired products in cell cultures or in a living body. Also provided are methods for eliciting an immune response in a subject in need thereof, as well as methods for preventing and/or treating various health conditions.

### CROSS-REFERENCE TO RELATED APPLICATIONS

This application claims the benefit of priority to U.S. Provisional Patent Application Ser. No. 63/240,297, filed on Sep. 2, 2021. The disclosure of the above-referenced application is herein expressly incorporated by reference it its entirety, including any drawings.

### FIELD

The present disclosure relates to the field of molecular virology and immunology, and particularly relates to nucleic acid molecules encoding modified viral genomes and replicons (e.g., self-replicating RNAs), pharmaceutical compositions containing the same, and the use of such nucleic acid molecules and compositions for production of desired products in cell cultures or in a living body. Also provided are methods for inducing pharmacodynamic effects, e.g., eliciting an immune response in a subject in need thereof, as well as methods for preventing and/or treating various health conditions.

### INCORPORATION OF THE SEQUENCE LISTING

The material in the accompanying Sequence Listing is hereby incorporated by reference into this application. The accompanying Sequence Listing XML file, named 058462_504001WO_SequenceListing.XML, was created on Aug. 28, 2022, and is 95 KB.

### BACKGROUND

In recent years, several different groups of animal viruses have been subjected to genetic manipulation either by homologous recombination or by direct engineering of their genomes. The availability of reverse genetics systems for both DNA and RNA viruses has created new perspectives for the use of recombinant viruses, for example, as vaccines, expression vectors, anti-tumor agents, gene therapy vectors, and drug delivery vehicles.

For example, many viral-based expression vectors have been deployed for expression of heterologous proteins in cultured recombinant cells. For example, the application of modified viral vectors for gene expression in host cells continues to expand. Recent advances in this regard include further development of techniques and systems for production of multi-subunit protein complexes, and co-expression of protein-modifying enzymes to improve heterologous protein production. Other recent progresses regarding viral expression vector technologies include many advanced genome engineering applications for controlling gene expression, preparation of viral vectors, in vivo gene therapy applications, and creation of vaccine delivery vectors.

Therefore, there is still a need for more efficient methods and systems for expressing products of interest in RNA replicon-based expression platforms.

### SUMMARY

The present disclosure relates generally to the development of immuno-therapeutics, such as recombinant nucleic acids constructs and pharmaceutical compositions including the same for use in the prevention and management of various health conditions such as proliferative disorders and microbial infection. In particular, as described in greater detail below, some embodiments of the disclosure provide, inter alia, nucleic acid constructs encoding recombinant alphavirus with a coding sequence for at least one heterologous one nonstructural protein (nsP) or a portion thereof. Also disclosed are recombinant cells containing the nucleic acid constructs disclosed herein, transgenic animal containing the nucleic acid constructs disclosed herein, methods for producing the nucleic acid constructed disclosed herein, methods for producing recombinant polypeptides of interest ex vivo or in vivo, and the recombinant polypeptides produced thereby, and pharmaceutical compositions containing the nucleic acid constructs, the recombinant cells, and the recombinant polypeptides. Also provided herein are methods for eliciting an immune response and methods for preventing and/or treating a health condition in a subject in need thereof, wherein the methods include administering the nucleic acid construct of the present disclosure, recombinant cells of the present disclosure, the recombinant polypeptides of the present disclosure, and/or the pharmaceutical composition of the present disclosure.

In one aspect of the disclosure, provided herein are nucleic acid constructs including a modified genome or RNA replicon (e.g., self-replicating RNA) of an alphavirus species, wherein at least one nonstructural protein (nsP), or a portion thereof, of the modified alphavirus genome or RNA replicon is heterologous relative to the remainder of the modified alphavirus genome or RNA replicon.

Non-limiting embodiments of the nucleic acid constructs of the disclosure can include one or more of the following features. In some embodiments, the at least one heterologous nsP or portion thereof is nsP1, nsP2, nsP3, nsP4, or a portion of any thereof, or a combination of any of the foregoing. In some embodiments, the at least one heterologous nsP or portion thereof is derived from another strain of the same alphavirus species. In some embodiments, the at least one heterologous nsP or portion thereof is derived from another alphavirus species.

In some embodiments, the modified alphavirus genome or RNA replicon (e.g., self-replicating RNA) is devoid of at least a portion of the nucleic acid sequence encoding one or more viral structural proteins. In some embodiments, the modified viral genome or RNA replicon is devoid of a substantial portion of the nucleic acid sequence encoding one or more viral structural proteins. In some embodiments, the modified viral genome or RNA replicon includes no nucleic acid sequence encoding viral structural proteins.

In some embodiments, the modified alphavirus genome or RNA replicon (e.g., self-replicating RNA) of the disclosure further includes one or more expression cassettes, wherein each of the expression cassettes includes a promoter operably linked to a heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, the modified alphavirus genome or RNA replicon of the disclosure further includes one or more untranslated regions (UTRs). In some embodiments, at least one of the UTRs is a heterologous UTR.

In some embodiments, at least one of expression cassettes includes a coding sequence for a gene of interest (GOI). In some embodiments, the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the coding sequence of the GOI is optimized for expression at a level higher than the expression level of a reference coding sequence. In some embodiments, the coding sequence of the GOI is optimized for enhanced RNA stability.

In some embodiments, the modified alphavirus genome or RNA replicon (e.g., self-replicating RNA) of the disclosure is of an alphavirus species selected from the group consisting of Aura virus (AURAV), Babanki virus (BABV), Barmah Forest virus (BFV), Bebaru virus (BEBV), Buggy Creek virus, Caaingua virus, Cabassou virus, Chikungunya virus (CHIKV), Eastern equine encephalitis virus (EEEV), Eilat virus, Everglades virus (EVEV), Fort Morgan virus (FMV), Getah virus (GETV), Highlands J virus (HJV), Kyzylagach virus (KYZV), Madariaga virus (MADV), Mayaro virus (MAYV), Middelburg virus (MIDV), Mosso das Pedras virus, Mucambo virus (MUCV), Ndumu virus (NDUV), O‘nyong’nyong virus (ONNV), Pixuna virus (PIXV), Rio Negro virus (RNV), Ross River virus (RRV), Salmon pancreas disease virus (SPDV), Semliki Forest virus (SFV), Sindbis virus (SINV), Sleeping disease virus (SDV), Southern elephant seal virus (SESV), Tai Forest virus (TFV), Tonate virus, Trocara virus, Una virus (UNAV), Venezuelan equine encephalitis virus (VEEV), Western equine encephalitis virus (WEEV), and Whataroa virus (WHAV).

In some embodiments, the modified alphavirus genome or RNA replicon (e.g., self-replicating RNA) of the disclosure is of a Sindbis virus (SINV). In some embodiments, the modified alphavirus genome or RNA replicon of the disclosure is of a SINV strain Girdwood.

In some embodiments, at least one heterologous nsP or portion thereof of the modified genome or RNA replicon (e.g., self-replicating RNA) is derived from a SINV strain AR86. In some embodiments, at least one heterologous nsP or portion thereof of the modified genome or RNA replicon is derived from a SINV strain Girdwood.

In some embodiments, the at least one heterologous nsP or portion thereof is nsP1, nsP3, nsP4, or a portion of any thereof, or a combination of any of the foregoing. In some embodiments, the modified genome or RNA replicon (e.g., self-replicating RNA) is of a SINV strain AR86. In some embodiments, the at least one heterologous nsP or portion thereof of the modified SINV-AR86 genome or RNA replicon is derived from a SINV strain Girdwood. In some embodiments, the at least one heterologous nsP or portion thereof of the modified SINV-AR86 genome or RNA replicon is derived from nsP2 of a SINV strain Girdwood.

In some embodiments, the nucleic acid construct of the disclosure is incorporated into a vector. In some embodiments, the vector is a self-replicating RNA (srRNA) vector.

In some embodiments of the disclosure, the nucleic acid construct including a nucleic acid sequence having at least 80%, at least 85%, at least 90%, at least 95%, at least 96%, at least 97%, at least 98%, at least 99%, or 100% sequence identity to a nucleic acid sequence selected from the group consisting of SEQ ID NOS: 1-4.

In one aspect, provided herein are recombinant cells including a nucleic acid construct as disclosed herein. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the animal cell is an insect cell. In some embodiments, the insect cell is a mosquito cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), canine kidney cell (MDCK), buffalo rat liver cell (BRL 3A), human lung cell (W138), human liver cell (Hep G2), mouse mammary tumor (MMT 060562), TRI cell, FS4 cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell. Also provided, in a related aspect, are cell cultures that include at least one recombinant cell as disclosed herein and a culture medium.

In another aspect, provided herein are transgenic animals including a nucleic acid construct as described herein. In some embodiments, the animal is a vertebrate animal or an invertebrate animal. In some embodiments, the animal is an insect. In some embodiments, the animal is a mammal. In some embodiments, the mammal is a non-human mammal. In another aspect, provided herein are methods for producing a polypeptide of interest, wherein the methods include (i) rearing a transgenic animal as disclosed herein; or (ii) culturing a recombinant cell including a nucleic acid construct as disclosed herein under conditions wherein the transgenic animal or recombinant cell produces the polypeptide encoded by the GOI.

In another aspect, provided herein are methods for producing a polypeptide of interest in a subject, wherein the methods include administering to the subject a nucleic acid construct as disclosed herein. In some embodiments, the subject is vertebrate animal or an invertebrate animal. In some embodiments, the subject is an insect. In some embodiments, the insect is a mosquito. In some embodiments, the subject is a mammalian subject. In some embodiments, the mammalian subject is a human subject. In yet another aspect, provided herein are recombinant polypeptides produced by a method of the disclosure.

In yet another aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient. In some embodiments, provided herein are compositions that formulated in a liposome, a lipid-based nanoparticle (LNP), or a polymer nanoparticle. In some embodiments, the compositions are immunogenic compositions. In some embodiments, the immunogenic compositions are formulated as a vaccine. In some embodiments, the immunogenic compositions are substantially non-immunogenic to a subject. In some embodiments, the pharmaceutical compositions are formulated as an adjuvant. In some embodiments, the pharmaceutical compositions are formulated for one or more of intranasal administration, intranodal administration, transdermal administration, intraperitoneal administration, intramuscular administration, intratumoral administration, intraarticular administration, intravenous administration, subcutaneous administration, intravaginal administration, intraocular administration, oral administration, and rectal administration.

In another aspect, provided herein are methods for functionalizing/engineering an alphavirus genome or RNA replicon (e.g., self-replicating RNA), the methods including: (a) providing a non-functional alphavirus genome or RNA replicon; (b) replacing a nonstructural protein (nsP), or a portion thereof, of the non-functional alphavirus genome or RNA replicon with a heterologous coding sequence for the corresponding nsP or portion thereof derived from a different alphavirus strain to generate a modified alphavirus genome or RNA replicon; (c) assessing functionality of the modified alphavirus genome or RNA replicon; and (d) identifying the modified alphavirus genome or RNA replicon as being functional if the modified alphavirus genome or RNA replicon is capable of RNA replication and/or expression.

Non-limiting exemplary embodiments of the p methods for functionalizing and/or engineering an alphavirus genome or RNA replicon (e.g., self-replicating RNA) of the disclosure can include one or more of the following features. In some embodiments, the heterologous nsP or portion thereof is derived from another strain of the same alphavirus species. In some embodiments, the heterologous nsP or portion thereof is derived from another alphavirus species. In some embodiments, the heterologous nsP or portion thereof is nsP1, nsP2, nsP3, nsP4, or a portion of any thereof. In some embodiments, the non-functionality of the alphavirus genome or RNA replicon is determined by a deficiency in self-replication within a host cell. In some embodiments, the assessing functionality of the modified alphavirus genome or RNA replicon includes an assay selected from the group consisting of: detection of RNA replication, detection of viral protein expression, detection of cytopathic effect (CPE), and detection of heterologous transgene expression.

In another aspect, provided herein are methods for inducing a pharmacodynamic effect in a subject and, in particular, methods for eliciting an immune response in a subject in need thereof, the methods include administering to the subject a composition including: a) a nucleic acid construct of the disclosure; b) a recombinant cell of the disclosure; c) a recombinant polypeptide of the disclosure; and/or d) a pharmaceutical composition of the disclosure.

In yet another aspect, provided herein are methods for preventing and/or treating a health condition in a subject in need thereof, the methods include prophylactically or therapeutically administering to the subject a composition including: a) a nucleic acid construct of the disclosure; b) a recombinant cell of the disclosure; c) a recombinant polypeptide of the disclosure; and/or d) a pharmaceutical composition of any one of the disclosure.

Non-limiting exemplary embodiments of the methods of the disclosure can include one or more of the following features. In some embodiments, the condition is a proliferative disorder or a microbial infection. In some embodiments, the subject has or is suspected of having a condition associated with proliferative disorder or a microbial infection. In some embodiments, the administered composition results in an increased production of interferon in the subject. In some embodiments, the composition is administered to the subject individually as a single therapy (monotherapy) or as a first therapy in combination with at least one additional therapies. In some embodiments, the at least one additional therapies is selected from the group consisting of chemotherapy, radiotherapy, immunotherapy, hormonal therapy, toxin therapy, targeted therapy, and surgery.

In yet another aspect, provided herein are kits for inducing a pharmacodynamic effect, for eliciting an immune response, for the prevention, and/or for the treatment of a health condition or a microbial infection, the kit including: a) a nucleic acid construct of the disclosure; b) a recombinant cell of the disclosure; c) a recombinant polypeptide of the disclosure; and/or d) a pharmaceutical composition of the disclosure.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a schematic representation of four non-limiting examples of alphavirus genome designs in accordance with some embodiments of the disclosure. Non-structural proteins nsP1, nsP2, nsP3, and nsP4 are shown. These alphavirus designs each contain (i) a heterologous gene of interest (GOI) placed under control of a 26S subgenomic promoter; and (ii) native 5′ UTR and 3′ UTR sequences derived from the SINV strain AR86. In AR86-Girdwood Hybrid 1, the structural proteins nsP1, nsP3, and nsP4 are from SINV strain AR86, while nsP2 is from SINV strain Girdwood. In AR86-Girdwood Hybrid 2, nsP4 is from AR86 strain, while nsP1, nsP2, and nsP3 are from Girdwood strain. In AR86-Girdwood Hybrid 3, nsP3 is from AR86 strain, whereas nsP1, nsP2, and nsP4 are from Girdwood strain. In AR86-Girdwood Hybrid 4, nsP1 are from AR86 strain, and nsP2, nsP3, whereas nsP4 is from Girdwood.

FIG. 2A is a schematic structure of the base Sindbis AR86-Girdwood Hybrid 1 vector described in FIG. 1 without coding sequence for a gene of interest (GOI). FIG. 2B is a schematic structure of the Sindbis AR86-Girdwood Hybrid 1 vector described in FIG. 1 with coding sequence for an exemplary GOI, e.g., hemagglutinin precursor (HA) of the influenza A virus H5N1 (H5N1 HA), which is placed under control of a 26S subgenomic promoter.

FIG. 3A is a schematic structures of the base Sindbis AR86-Girdwood Hybrid 2 vector described in FIG. 1 without coding sequence for a GOI. FIG. 3B is a schematic structures of the Sindbis AR86-Girdwood Hybrid 2 vector described in FIG. 1 with coding sequence for an exemplary GOI, e.g., H5N1 HA placed under control of a 26S subgenomic promoter.

FIG. 4A is a schematic structure of the base Sindbis AR86-Girdwood Hybrid 3 vector described in FIG. 1 without coding sequence for a GOI. FIG. 4B is a schematic structure of Sindbis AR86-Girdwood Hybrid 3 vectors described in FIG. 1 with coding sequence for an exemplary GOI, e.g., H5N1 HA placed under control of a 26S subgenomic promoter.

FIG. 5A is a schematic structure of Sindbis AR86-Girdwood Hybrid 4 vectors described in FIG. 1 without coding sequence for a GOI. FIG. 5B is a schematic structure of Sindbis AR86-Girdwood Hybrid 4 vectors described in FIG. 1, with coding sequence for an exemplary GOI, e.g., H5N1 HA placed under control of a 26S subgenomic promoter.

FIG. 6 graphically summarizes the results of experiments performed to demonstrate that a non-functional alphavirus genome or RNA replicon (e.g., self-replicating RNA) can be functionalized by replacing a defective nsP sequence with a corresponding functional nsP derived from a heterologous alphavirus genome or RNA replicon. FIG. 6 depicts contour plots of BHK-21 cells which have been transformed with exemplary alphavirus genome designs in accordance with some embodiments of the disclosure. In these experiments, the alphavirus genome designs were each introduced into BHK-21 cells by electroporation, and 20 hours following transformation, the cells were fixed and permeabilized and stained using a PE-conjugated anti-double stranded RNA (dsRNA) mouse monoclonal antibody (J2, Scicons) to quantify the frequency of dsRNA+ cells by fluorescence flow cytometry. The ability of the alphavirus genome designs to undergo RNA replication to result in production of dsRNA is indicated.

FIG. 7 graphically summarizes the results of experiments performed to demonstrate that expression of a GOI can be detected from srRNA vectors with heterologous nonstructural protein genes. FIG. 7 is a bar chart illustrating the quantification of relative expression of HA polypeptide of avian influenza A H5N1 in cells which have been transformed by srRNA vector designs in accordance with some embodiments of the disclosure. In these experiments, the alphavirus srRNA designs were each introduced into BHK-21 cells by electroporation, and 20 hours following transformation, the cells were fixed and permeabilized and stained using an APC-conjugated anti-H5N1 mouse monoclonal antibody (2B7, Abcam; APC: allophycocyanin) to quantify mean fluorescence intensity (MFI) of H5N1+ cells by fluorescence flow cytometry.

FIGS. 8A-8B schematically summarize the results of experiments demonstrating that modified srRNA vectors containing heterologous nonstructural protein genes designed in accordance with some embodiments of the disclosure can be used to express two exemplary bioactive proteins: (i) interleukin-1 receptor antagonist protein (IL-1RA) and (ii) interleukin-12 (IL-12). FIGS. 8A-8B are bar charts illustrating the quantification of secreted protein bioactivity from BHK-21 cells which were transformed with the srRNAs. The srRNAs shown in FIGS. 8A-8B are SINV AR86-Girdwood Hybrid 1 srRNAs (RBI307, RBI308) each encoding two proteins IL-1RA and IL-12 in two different configurations. Also included in these experiments were four control VEEV-based srRNAs as follows: VEEV srRNAs encoding both IL-1RA and IL-12 in two configurations (RBI299, RBI300) and VEEV srRNAs with control transgenes (RBI296, RBI298). Also included in these experiments were two control SINV Girdwood-based srRNAs (RBI309, RBI310) each encoding two proteins IL-1RA and IL-12 in two different configurations. FIG. 8A shows the quantification of bioactive IL-1RA in the cell culture media at 24 and 48 hours following srRNA transformation. FIG. 8B shows the quantification of bioactive IL-12 in the cell culture media at 24 and 48 hours following srRNA transformation.

FIGS. 9A-9B are bar charts illustrating in vivo immunogenicity of a panel of srRNAs encoding an exemplary viral antigen, which is an envelope glycoprotein G of a rabies virus (RABV-G). The panel included srRNAs derived from Venezuelan equine encephalitis virus (VEE.TC83), Chikungunya virus strains S27 (CHIK.S27) and DRDE-06 (CHIK.DRDE), Sindbis virus strains Girdwood (SIN.GW) and AR86-Girdwood Hybrid 1 (SIN.AR86), and Eastern equine encephalitis virus (EEE.FL93). FIG. 9A shows the quantification of antigen-specific splenic T cell responses evaluated by ELISpot after two immunizations. FIG. 9B shows anti-rabies neutralizing antibody titers from sera after two immunizations.

FIGS. 10A-10C are bar charts showing in vivo immunogenicity of a panel of srRNAs encoding exemplary tumor-associated antigens for use as vaccine, e.g., for eliciting an immune response in a subject. The panel included srRNAs derived from Sindbis AR86-Girdwood Hybrid 1 (SIN.AR86) and five other alphaviruses: Venezuelan equine encephalitis virus (VEE.TC83), Chikungunya virus strains S27 (CHIK.S27) and DRDE-06 (CHIK.DRDE), Sindbis virus strain Girdwood (SIN.GW) and Eastern equine encephalitis virus (EEE.FL93). Each srRNA includes sequences encoding for three polypeptides: sequence for estrogen receptor 1 (ESR1), human epidermal growth factor 2_(HER2), and human epidermal growth factor 2 (HER3). FIGS. 10A-10C show splenic T cell responses to these three antigens determined using ELISpot analysis in mice having received two immunizations, with statistical comparisons between each antigen tested.

### DETAILED DESCRIPTION OF THE DISCLOSURE

Provided herein are, inter alia, viral expression systems with superior expression potential which are suitable for expressing recombinant polypeptides such as, for example, vaccines and therapeutic polypeptides, in recombinant cells. For example, some embodiments of the disclosure relate to nucleic acid constructs such as, e.g., expression constructs and vectors, containing a modified genome or replicon RNA (e.g., self-replicating RNA) of an alphavirus species, wherein at least one nonstructural protein (nsP), or a portion thereof, of the modified alphavirus genome or RNA replicon is heterologous relative to the remainder of the modified alphavirus genome or RNA replicon. Also provided in some embodiments of the disclosure are viral-based expression vectors including one or more expression cassettes encoding a polypeptide of interest encoded by a gene of interest (GOI). Further provided are recombinant cells that are genetically engineered to include one or more of the nucleic acid constructs disclosed herein. Biomaterials and recombinant products derived from such recombinant cells are also within the scope of the application. Also provided are compositions and methods useful inducing pharmacodynamic effects, e.g., for eliciting an immune response, in a subject in need thereof, as well as methods for preventing and/or treating various health conditions.

Self-amplifying RNAs (e.g., self-replicating RNA or replicons) based on RNA viruses (e.g., alphaviruses) can be used as robust expression systems. For example, it has been reported that an advantage of using alphaviruses such as SINV as viral expression vectors is that they can direct the synthesis of large amounts of recombinant proteins in recombinant host cells. Among other advantages, polypeptides such as therapeutic single chain antibodies can be most effective if expressed at high levels in vivo. In addition, for producing recombinant antibodies purified from cells in culture (ex vivo), high protein expression from a replicon RNA can increase overall yields of the antibody product. Furthermore, if the protein being expressed is a vaccine antigen, high level expression can induce the most robust immune response in vivo.

Alphaviruses utilize motifs contained in their UTRs, structural regions, and non-structural regions to impact their replication in host cells. These regions also contain mechanism to evade host cell innate immunity. However, significant differences among alphavirus species have been reported, for example in mechanisms of immune evasion, tissue tropism, xenotropic hosts, as well as disease symptoms and severity.

Given the differential presence of host cell attenuating factors in non-structural and structural regions of Alphaviruses, deleting the structural genes to allow for expression of a gene of interest expression in synthetic vectors will have varied impacts on individual vectors. Synthetic replicons (e.g., self-replicating RNAs) with different host attenuating factors in the non-structural regions will differentially excel at the induction of immune responses to the expressed gene of interest. The avirulent Sindbis Girdwood strain's inability to inhibit STAT1 makes it an advantaged vector for expression of recombinant proteins without forming robust immune responses against the encoded protein. The advantages that these individual vectors confer has been up until now completely unexplored and unpredicted.

As described in greater detail below, an initial observation was made that the publicly available alphavirus genomic data does not always provide nucleotide sequences that are capable of direct replacement of the nucleic acid sequences encoding the structural proteins with a gene of interest (GOI) to result in self-replicating RNA and transgene-expressing replicons. In particular, a large number of publicly available alphavirus genomes were found non-functional, e.g., incapable of undergoing replication and/or expressing a transgene. As described in greater detail in the working examples below, provided herein is, inter alia, a new procedure useful for functionalizing a defective (e.g., non-functional) alphavirus genome or RNA replicon (e.g., self-replicating RNA) by replacing a nonstructural protein (nsP) sequence of the defective alphavirus genome or RNA replicon with the corresponding nsP sequence derived from a different alphavirus strain to generate a modified alphavirus genome or RNA replicon which is functional.

It has not been fully appreciated that full-length viruses and synthetic replicons (e.g., self-replicating RNAs) do not have the same capacity for replication. In particular, many full-length viruses and replicons from publicly available resources are functionally defective. Currently, a modification approach to functionalize (e.g., to render functional) a defective alphavirus genome or RNA replicon is to revert one or more key point mutations related to virulence that diverged between strains, for example mutations that diverse between a functional strain (e.g., Girdwood) and a non-functional strain (e.g., AR86). However, this strategy often fails or a solution is arrived at arbitrarily, indicating that there is far more uncharacterized and thus unpredictable sequence divergence between strains. Therefore, there is a need for more rapid and efficient way to identify functional alphavirus strains (instead of simply reverting point mutations or selecting arbitrary regions to generate chimeras).

As described above, during viral replication, each of the nsP subunits (e.g., nsP1, nsP2, nsP3, nsP4) of an nsP polyprotein complex is processed separately into an individual protein. These proteins then come together to form an nsP polyprotein complex that performs genomic and subgenomic transcription function. Without being bound to any particular theory, it is hypothesized that each nsP itself is reasonably biologically self-contained in terms of contributing to the overall function of the replicon (e.g., self-replicating RNA) and should be treated as a discrete modular unit. As described in greater detail below, some embodiments of the present disclosure relate to a method of functionalizing a non-functional alphavirus genome or RNA replicon, wherein each of the nsP subunits is treated as a discrete modular unit and can be replaced (swapped) by a corresponding modular unit from another virus (e.g., another species or another strain of the same species), resulting in a chimeric virus with new characteristics. The presently disclosed method allows for a new combinatorial look at the effect of swapping out nsPs in a following minimal set: (1) a replicon that does not launch in vitro; and (2) a replicon that does launch in vitro. This approach rapidly gives information on which nsP is creating issues for any given strain without the need to create a large number of new constructs. Thus, the presently described new procedure is a rapid, technically feasible method that is significantly improved than any of the currently known methods.

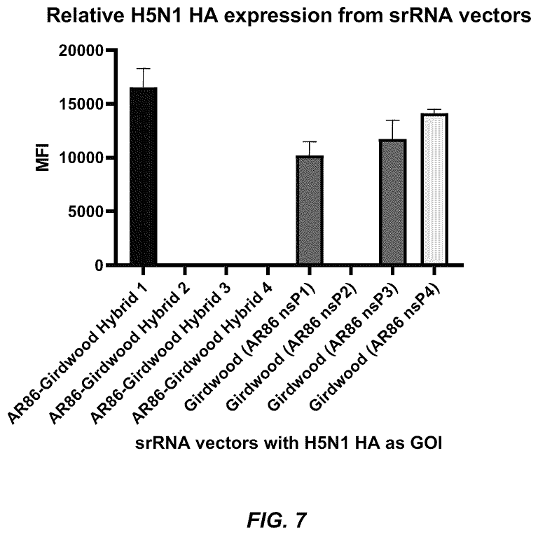

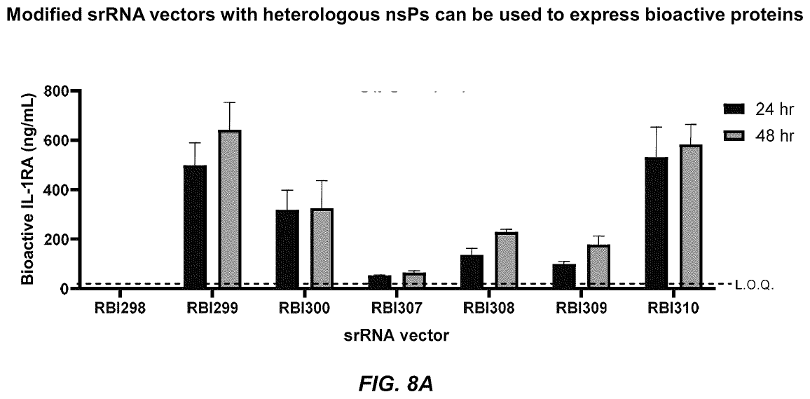

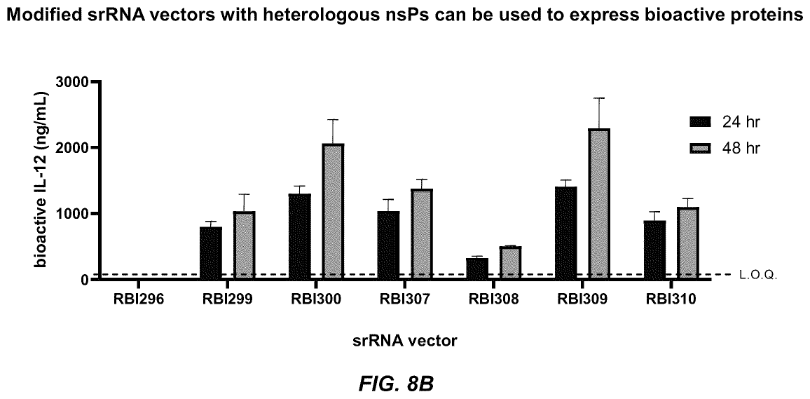

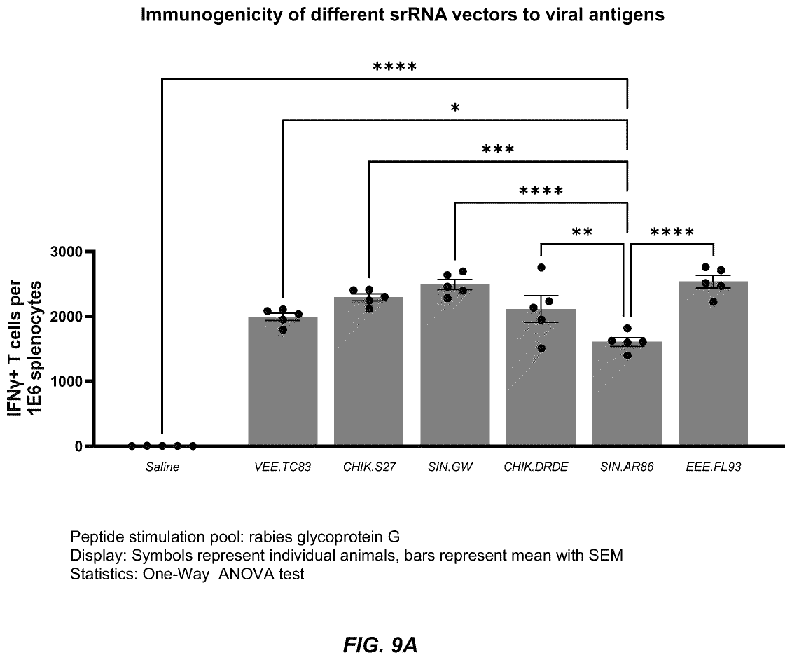

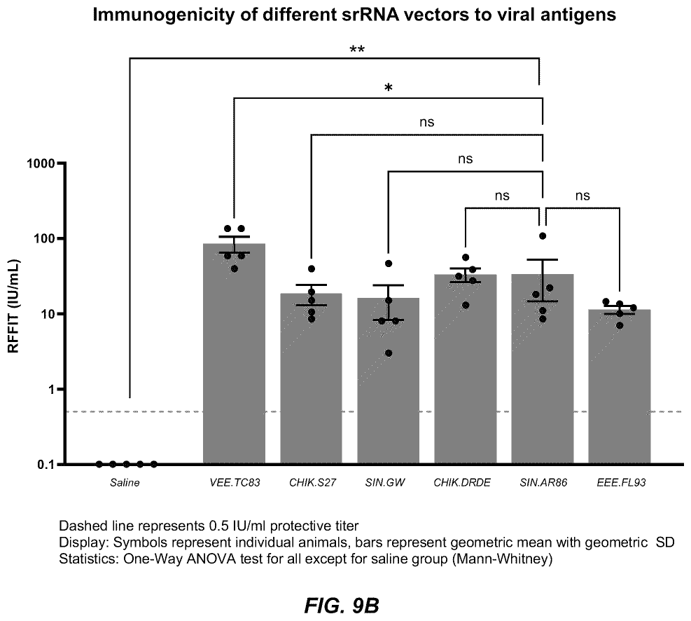

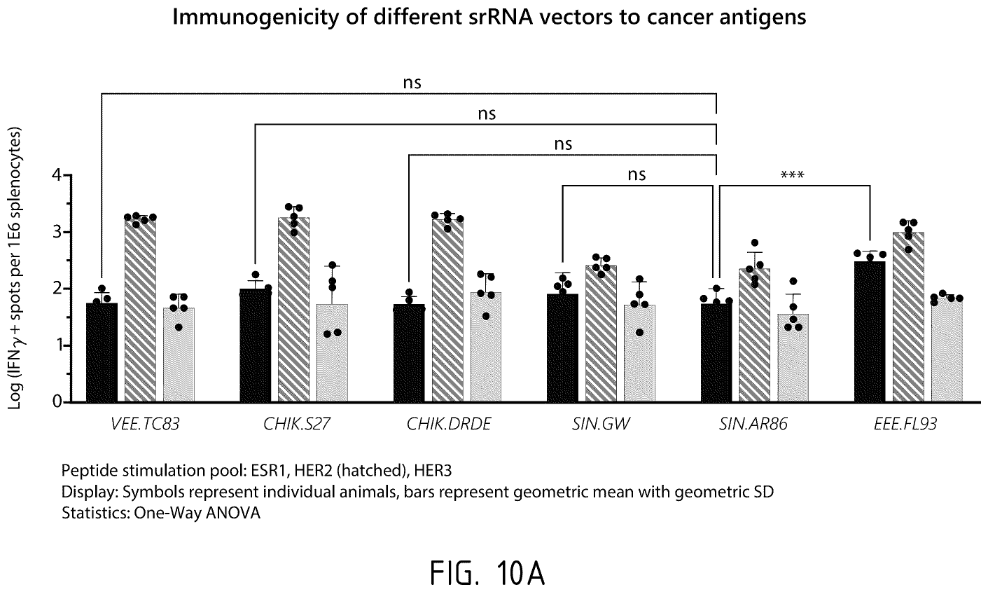

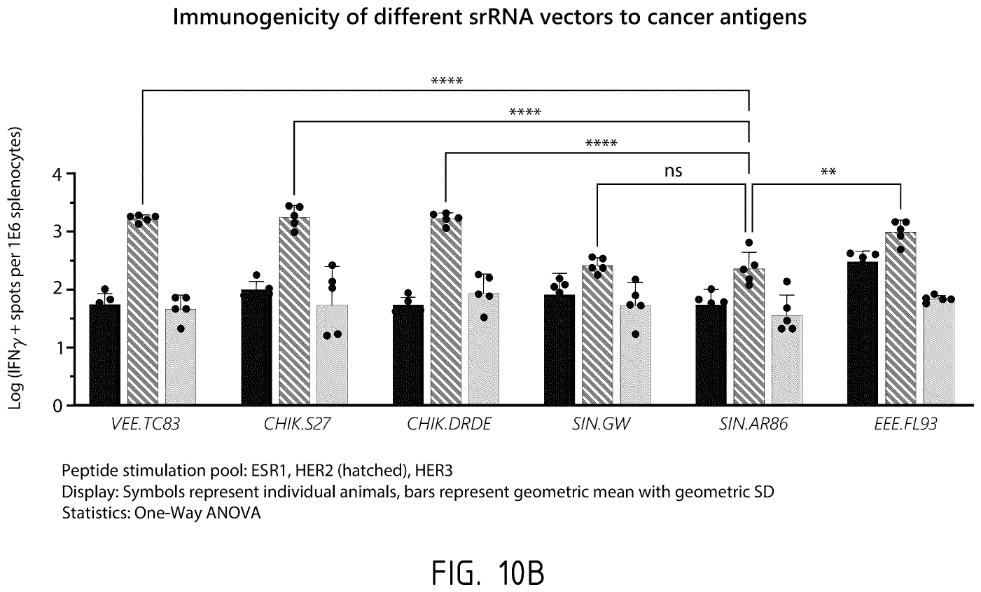

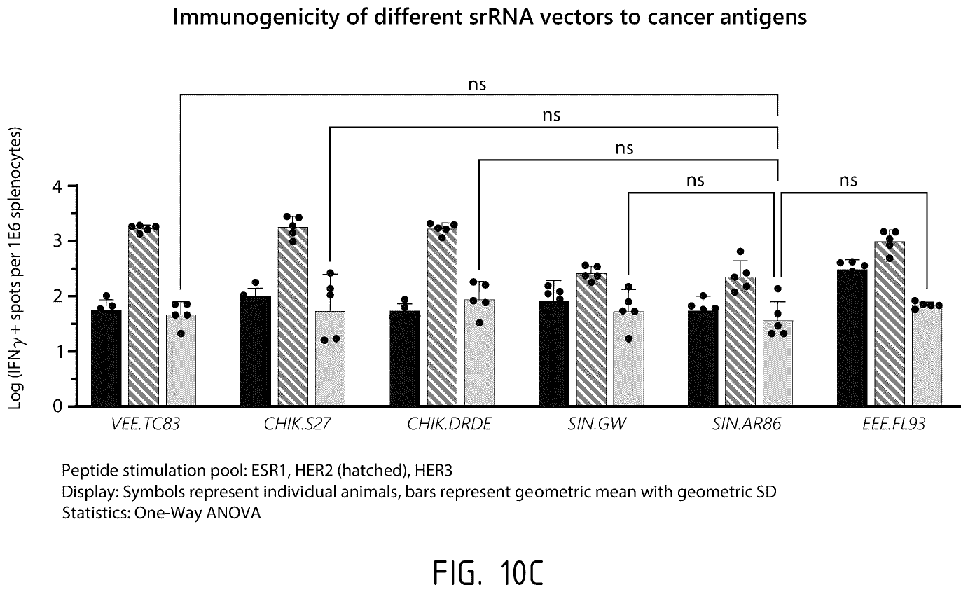

As described in greater detail below, some embodiments of the disclosure relate to self-replicating RNA (srRNAs) vectors containing heterologous nonstructural protein genes that have been engineered to express one or more heterologous genes of interest (GOI). For example, it was found possible to replace the structural polyprotein gene in srRNA vectors containing one or more heterologous nonstructural protein genes with one or more GOIs. In one exemplification, a Sindbis srRNA vector containing heterologous nonstructural protein genes (SINV AR86-Girdwood Hybrid 1) has been engineered to replace the structural polyprotein gene with a synthetic human IL-1RA gene or IL-12 gene cassette to produce self-replicating vectors capable of RNA replication and transgene expression in transfected BHK-21 cells (see e.g., FIG. 8). In addition, as described in greater detail below, some SINV AR86-Girdwood Hybrid 1-based srRNA constructs as described herein can be employed for expression of an antigenic molecule of interest and formulated as a vaccine with a measurable pharmacodynamic effect in vivo (see, e.g., FIG. 9 and FIG. 10). Furthermore, experimental data described in FIGS. 7A-7B demonstrate that SINV AR86-Girdwood Hybrid 1-based srRNA vectors can be useful for expression of multiple proteins whose coding sequences are operably linked to one another within a single open reading frame (e.g., in a polycistronic ORF) and have bioactivity as measured by pharmacodynamic effect in vivo (see, e.g., FIG. 10). Taken together, these studies further demonstrate the use of srRNA vectors with heterologous nonstructural protein genes and SINV AR86-Girdwood Hybrid 1-based srRNA vectors in therapeutic and vaccine applications.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

The terms “administration” and any grammatical variation thereof, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications can occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a composition of the disclosure, e.g., nucleic acid constructs, recombinant cells, recombinant polypeptides, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “nucleic acid construct” refers to a recombinant nucleic acid molecule including one or more isolated nucleic acid sequences from heterologous sources. For example, nucleic acid constructs of the disclosure can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single-stranded or double-stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Constructs of the present disclosure can include the necessary elements to direct expression of a nucleic acid sequence of interest that is also contained in the construct. Such elements can include control elements such as a promoter that is operably linked to (so as to direct transcription of) the nucleic acid sequence of interest, and optionally includes a polyadenylation sequence.

In some embodiments of the disclosure, the nucleic acid construct can be incorporated within a vector. The term “vector” is used herein to refer to a nucleic acid molecule or sequence capable of transferring or transporting another nucleic acid molecule. Thus, the term “vector” encompasses both DNA-based vectors and RNA-based vectors. The term “vector” includes cloning vectors and expression vectors, as well as viral vectors and integrating vectors. An “expression vector” is a vector that includes a regulatory region, thereby capable of expressing DNA sequences and fragments in vitro, ex vivo, and/or in vivo. In some embodiments, a vector may include sequences that direct autonomous replication in a cell such as, for example a plasmid (DNA-based vector) or a self-replicating RNA vector. In some embodiments, a vector may include sequences sufficient to allow integration into host cell DNA. In some embodiments, a vector may include DNA sequences that can be transcribed into RNA in vitro and/or in vivo. Useful vectors include, for example, plasmids (e.g., DNA plasmids or RNA plasmids), transposons, cosmids, bacterial artificial chromosomes, and viral vectors. In some embodiments, the vector of the disclosure can be single-stranded vector (e.g., ssDNA or ssRNA). In some embodiments, the vector of the disclosure can be double-stranded vector (e.g., dsDNA or dsRNA). In some embodiments, a vector is a gene delivery vector. In some embodiments, a vector is used as a gene delivery vehicle to transfer a gene into a cell.

In addition to the components of the construct, the vector can include, for example, one or more selectable markers, one or more origins of replication, such as prokaryotic and eukaryotic origins, at least one multiple cloning site, and/or elements to facilitate stable integration of the construct into the genome of a cell. Two or more constructs can be incorporated within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in a cell. For the practice of the present disclosure, compositions and methods for preparing and using constructs and cells are known to one skilled in the art.

The term “effective amount”, “therapeutically effective amount”, or “pharmaceutically effective amount” of a composition of the disclosure, e.g., nucleic acid constructs, recombinant cells, recombinant polypeptides, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements can be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein can be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60%, about 65%, about 70%, about 75%, about 80%, about 85%, about 90%, about 91%, about 92%, about 93%, about 94%, about 95%, about 96%, about 97%, about 98%, about 99%, or higher sequence identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI website at ncbi.nlm.nih.gov/BLAST. Such sequences are then said to be “substantially identical.” This definition also refers to, or can be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “portion” as used herein can refer to a fraction. With respect to a particular structure such as an amino acid sequence or protein, the term “portion” thereof can designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure, each part being a continuous element of the structure. For example, a discontinuous fraction of an amino acid sequence can be composed of 2, 3, 4, 5, 6, 7, 8, or more, for example not more than 4 parts of said amino acid sequence, wherein each part comprises at least 2, 3, 4, 5 continuous amino acids, at least 10 continuous amino acids, at least 20 continuous amino acids, at least 30 continuous amino acids of the amino acid sequence.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, the term “replicon RNA” or “RNA replicon” refers to RNA which contains all of the genetic information required for directing its own amplification or self-replication within a permissive cell. Therefore, replicon RNA is sometimes also referred to as “self-amplifying RNA” (saRNA) or “self-replicating RNA” (srRNA). To direct its own replication, the RNA molecule 1) encodes polymerase, replicase, or other proteins which may interact with viral or host cell-derived proteins, nucleic acids or ribonucleoproteins to catalyze the RNA amplification process; and 2) contain cis-acting RNA sequences required for replication and transcription of the subgenomic replicon-encoded RNA. These sequences may be bound during the process of replication to its self-encoded proteins, or non-self-encoded cell-derived proteins, nucleic acids or ribonucleoproteins, or complexes between any of these components. For the purpose of the present disclosure, an alphavirus replicon RNA molecule (e.g., srRNA or saRNA molecule) generally contains the following ordered elements: 5′ viral RNA sequence(s) required in cis for replication, sequences coding for biologically active alphavirus non-structural proteins (e.g., nsP1, nsP2, nsP3, and nsP4), promoter for the subgenomic RNA (sgRNA), 3′ viral sequences required in cis for replication, and a polyadenylate tract (poly(A)). Further, the term replicon RNA (e.g., srRNA or saRNA molecule) generally refers to a molecule of positive polarity, or “message” sense, and the replicon RNA may be of length different from that of any known, naturally-occurring alphavirus. In some embodiments of the present disclosure, the replicon RNA does not contain the sequences of at least one of structural viral protein; and/or sequences encoding structural genes can be substituted with heterologous sequences. In those instances, where the replicon RNA is to be packaged into a recombinant alphavirus particle, it can contain one or more sequences, so-called packaging signals, which serve to initiate interactions with alphavirus structural proteins that lead to particle formation.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer or infection) and/or one or more symptoms of the health condition. The subject can also be an individual who is diagnosed with a risk of the health condition and/or disease of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood by one having ordinary skill in the art that all ranges disclosed herein encompass any and all possible sub-ranges and combinations of sub-ranges thereof Δny listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. In addition, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about” which, as used herein, has its ordinary meaning of approximately. The term “about” is used to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Alphaviruses

Alphaviruses are small, enveloped RNA viruses with a single-stranded, positive-sense RNA genome. The alphavirus genus includes, inter alia, the Sindbis virus (SINV), the Semliki Forest virus (SFV), the Ross River virus (RRV), Venezuelan equine encephalitis virus (VEEV), and Eastern equine encephalitis virus (EEEV), which are all closely related and are able to infect various vertebrates such as mammalians, rodents, fish, avian species, and larger mammals such as humans and horses as well as invertebrates such as insects. In particular, the Sindbis and the Semliki Forest viruses have been widely studied and the life cycle, mode of replication, etc., of these viruses are well characterized. Non-limiting exemplary alphavirus species include Aura virus (AURAV), Babanki virus (BABV), Barmah Forest virus (BFV), Bebaru virus (BEBV), Buggy Creek virus, Caaingua virus, Cabassou virus, Chikungunya virus (CHIKV), Eastern equine encephalitis virus (EEEV), Eilat virus, Everglades virus (EVEV), Fort Morgan virus (FMV), Getah virus (GETV), Highlands J virus (HJV), Kyzylagach virus (KYZV), Madariaga virus (MADV), Mayaro virus (MAYV), Middelburg virus (MIDV), Mosso das Pedras virus, Mucambo virus (MUCV), Ndumu virus (NDUV), O‘nyong’nyong virus (ONNV), Pixuna virus (PIXV), Rio Negro virus (RNV), Ross River virus (RRV), Salmon pancreas disease virus (SPDV), Semliki Forest virus (SFV), Sindbis virus (SINV), Sleeping disease virus (SDV), Southern elephant seal virus (SESV), Tai Forest virus (TFV), Tonate virus, Trocara virus, Una virus (UNAV), Venezuelan equine encephalitis virus (VEEV), Western equine encephalitis virus (WEEV), and Whataroa virus (WHAV).

The alphavirus genome is approximately 12 kb long, and it consists of two open reading frames (ORFs): a 7 kb frame encoding the nonstructural proteins (nsPs) and a 4 kb frame encoding the structural polyprotein. The non-structural polyprotein (nsP) is cleaved into four different proteins (nsP1, nsP2, nsP3, and nsP4) which are necessary for the transcription and translation of viral mRNA inside the cytoplasm of host cells.

The nsP1 protein is an mRNA capping enzyme that possesses both guanine-7-methyltransferase (MTase) and guanylyltransferase (GTase) activities, where they direct the methylation and capping of newly synthesized viral genomic and subgenomic RNAs. The MTase motif in the N-terminal domain of nsP1 catalyzes the transfer of the methyl group from S-adenosylmethionine (AdoMet) to the N7 position of a GTP molecule (m7Gppp). GTase then binds the m7Gppp, forming a covalent link with a catalytic histidine (m7Gp-GTase) and releasing PPi. The GTase then transfers the m7Gp molecule to the 5′-diphosphate RNA to create m7GpppNp-RNA. The resulting cap structure is essential for viral mRNA translation and prevents the mRNA from being degraded by cellular 5′ exonucleases. Following the N-terminal domain are features that allow the association of the nsP1 protein to cellular membranes. The presence of α-helical amphipathic loop and palmitoylation sites allow the nsP1 protein and nsP1-containing replication complex to anchor onto the plasma membrane, possibly through nsP1 interaction with the membrane's anionic phospholipids.

The nsP2 protein possesses numerous enzymatic activities and functional roles. The N-terminal region contains a helicase domain that has seven signature motif of Superfamily 1 (SF1) helicases. It functions as an RNA triphosphatase that performs the first of the viral RNA capping reactions. It also functions as a nucleotide triphosphatase (NTPase), fueling the RNA helicase activity. The C-terminal region of nsP2 contains a papain-like cysteine protease, which is responsible for processing the viral non-structural polyprotein. The protease recognizes conserved motifs within the polyprotein. This proteolytic function is highly regulated and is modulated by other domains of nsP2. The alphavirus nsP2 protein has also been described as a virulence factor responsible for the transcriptional and translational shutoff in infected host cells and the inhibition of interferon (IFN) mediated antiviral responses contributing to the controlling of translational machinery by viral factors.

The precise role(s) of alphavirus nsP3 protein in the replication complex is less clear. The nsP3 protein has three recognized domains: the N-terminal macrodomain with phosphatase activity and nucleic acid binding ability, the alphavirus unique domain (AUD) and the C-terminal hypervariable domain. It has been demonstrated that the deletion of this domain in SFV nsP3 resulted in low viral pathogenicity, suggesting its importance in viral RNA transcription regulation.

The nsP4 polymerase is the most highly conserved protein in alphaviruses, with the most divergent being >50% identical in amino acid sequence when compared with other alphaviral nsP4s. The nsP4 contains the core RNA-dependent RNA polymerase (RdRp) domain at the C-terminal end, determined to be solely responsible for the RNA synthetic properties of the viral replication complex. The RdRp participates in replicating the genomic RNA via a negative strand RNA and transcribing the 26S subgenomic RNA. The N-terminal domain is alphavirus-specific and can be partially disordered structurally.

The 5′ two-thirds of the alphavirus genome encodes a number of nonstructural proteins (nSPs) necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1, nsP2, nsP3, nsP4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The subgenomic RNA is transcribed from the p26S subgenomic promoter present at the 3′ end of the RNA sequence encoding the nsP4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsP and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

### Compositions of the Disclosure

As described in greater detail below, one aspect of the present disclosure relates to nucleic acid constructs a nucleic acid sequence encoding a modified viral genome or replicon RNA (e.g., self-replicating RNA) of an alphavirus species, a recombinant cell comprising the nucleic acid construct, a transgenic animal comprising the nucleic acid construct, and a recombinant polypeptide produced by the methods of the present disclosure.

Some embodiments of the disclosure provide a modified alphavirus genome or replicon RNA (e.g., self-replicating RNA) in which at least one structural protein (nsP), or a portion thereof, of the modified alphavirus genome or RNA replicon is heterologous relative to the remainder of the modified alphavirus genome or RNA replicon, wherein the at least one heterologous nsP or portion thereof is nsP1, nsP2, nsP3, nsP4, or a portion of any thereof, or a combination of any of the foregoing. In some embodiments, nsP1 or a portion thereof is heterologous relative to the remainder of the modified alphavirus genome or RNA replicon. In some embodiments, nsP2 or a portion thereof is heterologous relative to the remainder of the modified alphavirus genome or RNA replicon. In some embodiments, nsP3 or a portion thereof is heterologous relative to the remainder of the modified alphavirus genome or RNA replicon. In some embodiments, nsP4 or a portion thereof is heterologous relative to the remainder of the modified alphavirus genome or RNA replicon. In some embodiments, two nsP proteins are heterologous relative to the remainder of the modified alphavirus genome or RNA replicon. In some embodiments, three nsP proteins are heterologous relative to the remainder of the modified alphavirus genome or RNA replicon. In some embodiments, nsP1, nsP2, and nsP3 or a portion thereof are heterologous relative to the remainder of the modified alphavirus genome or RNA replicon. In some embodiments, nsP1, nsP2, and nsP4 or a portion thereof are heterologous relative to the remainder of the modified alphavirus genome or RNA replicon. In some embodiments, nsP2, nsP3, and nsP4 or a portion thereof are heterologous relative to the remainder of the modified alphavirus genome or RNA replicon.

### A. Nucleic Acid Constructs

As described in greater detail below, one aspect of the present disclosure relates to novel nucleic acid constructs including a nucleic acid sequence encoding a modified genome or replicon RNA (e.g., self-replicating RNA) of an alphavirus, such as Sindbis virus (SINV), wherein at least one nonstructural protein (nsP), or a portion thereof, of the modified alphavirus genome or RNA replicon is heterologous relative to the remainder of the modified alphavirus genome or RNA replicon. For example, the at least one heterologous nsP or portion thereof is nsP1, nsP2, nsP3, nsP4, or a portion of any thereof, or a combination of any of the foregoing. As described above, the skilled artisan will understand that a portion of a nucleic acid sequence encoding a nonstructural polypeptide can include enough of the nucleic acid sequence encoding the nonstructural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a portion of a nucleotide sequence comprises enough of the sequence to afford specific identification and/or isolation of a nucleic acid fragment comprising the sequence. For example, a portion of a nucleic acid sequence can include at least about 20%, for example, about 30%, about 40%, about 50%, about 60%, about 70%, about 80%, about 90%, about 95% of the full-length nucleic acid sequence.

Non-limiting exemplary alphavirus species suitable for the compositions and methods of the present disclosure include Aura virus (AURAV), Babanki virus (BABV), Barmah Forest virus (BFV), Bebaru virus (BEBV), Buggy Creek virus, Caaingua virus, Cabassou virus, Chikungunya virus (CHIKV), Eastern equine encephalitis virus (EEEV), Eilat virus, Everglades virus (EVEV), Fort Morgan virus (FMV), Getah virus (GETV), Highlands J virus (HJV), Kyzylagach virus (KYZV), Madariaga virus (MADV), Mayaro virus (MAYV), Middelburg virus (MIDV), Mosso das Pedras virus, Mucambo virus (MUCV), Ndumu virus (NDUV), O‘nyong’nyong virus (ONNV), Pixuna virus (PIXV), Rio Negro virus (RNV), Ross River virus (RRV), Salmon pancreas disease virus (SPDV), Semliki Forest virus (SFV), Sindbis virus (SINV), Sleeping disease virus (SDV), Southern elephant seal virus (SESV), Tai Forest virus (TFV), Tonate virus, Trocara virus, Una virus (UNAV), Venezuelan equine encephalitis virus (VEEV), Western equine encephalitis virus (WEEV), and Whataroa virus (WHAV). Virulent and avirulent alphavirus strains are both suitable. In some embodiments, the alphavirus is Venezuelan equine encephalitis virus (VEEV). In some embodiments, the alphavirus is Eastern Equine Encephalitis virus (EEEV). In some embodiments, the alphavirus is Western Equine Encephalitis virus (WEEV).

In some embodiments, the alphavirus is Chikungunya virus (CHIKV). Non-limiting examples of CHIKV strains suitable for the compositions and methods of the disclosure include CHIKV S27, CHIKV LR2006-OPY-1, CHIKV Y0123223, CHIKV DRDE, CHIKV 37997, CHIKV 99653, CHIKV Ag41855, and Nagpur (India) 653496 strain. Virulent and avirulent CHIKV strains are both suitable. Additional examples of CHIKV strains suitable for the compositions and methods of the disclosure include but are not limited to those described in Afreen et al. Microbiol. Immunol. 2014, 58:688-696, Lanciotti and Lambert ASTMH 2016, 94(4):800-803 and Langsjoen et al. mBio. 2018, 9(2):e02449-17. In some embodiments, the modified CHIKV genome or replicon RNA (e.g., self-replicating RNA) is derived from CHIKV strain S27. In some embodiments, the modified CHIKV genome or replicon RNA is derived from CHIKV strain DRDE. In some embodiments, the modified CHIKV genome or replicon RNA is derived from CHIKV strain DRDE-06. In some embodiments, the modified CHIKV genome or replicon RNA is derived from CHIKV strain DRDE-07.

In some embodiments, the alphavirus is Eastern Equine Encephalitis virus (EEEV). Non-limiting examples of EEEV strains suitable for the compositions and methods of the disclosure include EEEV 792138, 783372, BeAn5122, BeAr300851, BeAr436087, C-49, FL91-4679, FL93-939, GML903836, MP-9, PE6, and V105-00210. Virulent and avirulent EEEV strains are both suitable. Additional suitable EEEV strains include, but are not limited to those described in the Virus Pathogen Resource website (ViPR; which is publicly available at www.viprbrc.org/brc/vipr_genome_search.spg?method=SubmitForm&blockId=868&decorator=toga). In some embodiments, the modified EEEV genome or replicon RNA (e.g., self-replicating RNA) is derived from EEEV strain FL93-939.

In some embodiments, the alphavirus is Sindbis virus (SINV). In some embodiments, the modified genome or RNA replicon (e.g., self-replicating RNA) is of a SINV strain. Non-limiting examples of SINV strains suitable for the compositions and methods of the disclosure include SINV strain AR339, AR86, and Girdwood. Examples of SINV strains suitable for the compositions and methods of the disclosure include, but are not limited to those described in Sammels et al. J. Gen. Virol. 1999, 80(3):739-748, Lundstrom and Pfeffer Vector Borne Zoonotic Dis. 2010, 10(9):889-907, Sigei et al. Arch. of Virol. 2018, 163:2465-2469 and Ling et al. J. Virol. 2019, 93:e00620-19. Additional suitable SINV strains include, but are not limited to those described in the Virus Pathogen Resource website (ViPR; which is publicly available at www.viprbrc.org/brc/vipr_genome_search.spg?method=SubmitForm&blockId=868&decorator=toga). Virulent and avirulent SINV strains are both suitable. In some embodiments, the modified genome or RNA replicon is of a SINV strain Girdwood. In some embodiments, the modified genome or RNA replicon is of a SINV strain AR86. In some embodiments, the modified SINV genome or replicon RNA is derived from SINV strain Girdwood. In some embodiments, the modified SINV genome or replicon RNA is derived from SINV strain AR86. In some embodiments, the at least one heterologous nsP or portion thereof of the modified genome or RNA replicon is derived from a SINV strain AR86. In some embodiments, the at least one heterologous nsP or portion thereof is nsP1, nsP3, nsP4, or a portion of any thereof, or a combination of any of the foregoing. In some embodiments, the modified genome or RNA replicon is of a SINV strain AR86.

In some embodiments, the alphavirus is Western Equine Encephalitis virus (WEEV). Non-limiting examples of WEEV strains suitable for the compositions and methods of the disclosure include WEEV California, McMillan, IMP181, Imperial, Imperial181, IMPR441, 71V-1658, AG80-646, BFS932, COA592, EP-6, E1416, BFS1703, BFS2005, BSF3060, BSF09997, CHLV53, KERN5547, 85452NM, Montana-64, S8-122, and TBT-235. Additional examples of WEEV strains suitable for the compositions and methods of the disclosure include 5614, 93A27, 93A30, 93A38, 93A79, B628(Cl 15), CBA87, CNTR34, C0921356, Fleming, Lake43, PV012357A, PV02808A, PV72102, R02PV001807A, R02PV002957B, R02PV003422B, R05PV003422B, ROPV003814A and ROPV00384A. Virulent and avirulent WEEV strains are both suitable. Additional suitable WEEV strains include, but are not limited to those described in Bergren N A et al., J. Virol. 88(16): 9260-9267, August 2014, and in the Virus Pathogen Resource website (ViPR; which is publicly available at https://www.viprbrc.org/brc/vipr_genome_search.spg?method=SubmitForm&blockId=57240&d ecorator-toga). In some embodiments, the modified WEEV genome or srRNA is derived from WEEV strain Imperial. In some embodiments, the modified WEEV genome or srRNA is derived from WEEV strain McMillan.

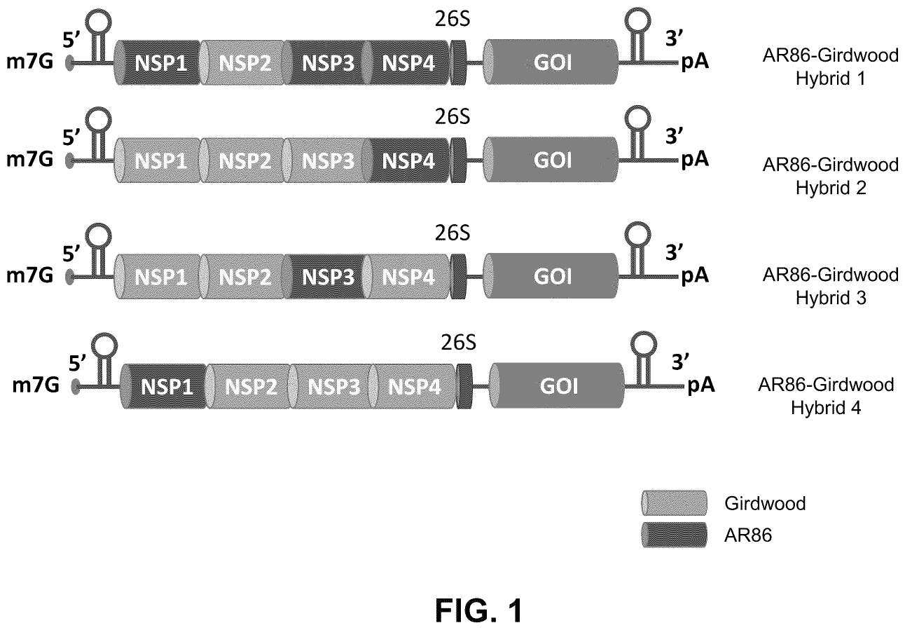

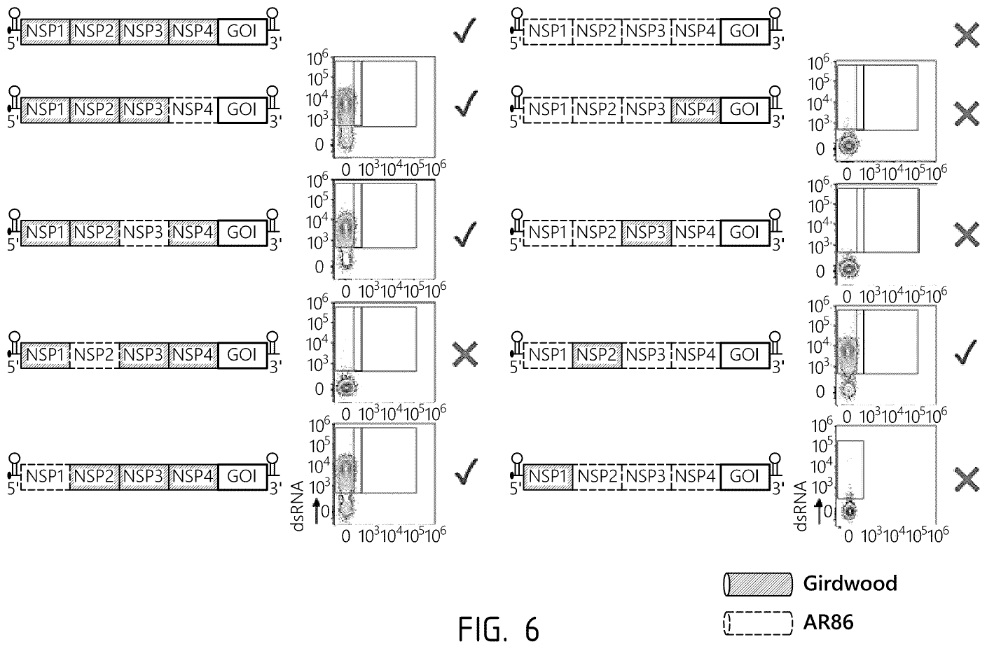

In some embodiments, at least one heterologous nsP or a portion thereof is derived from another strain of the same alphavirus species. In some embodiments, at least one heterologous nsP or portion thereof is derived from another alphavirus species. For example, in some embodiments, at least one heterologous nsP or portion thereof of the modified SINV-AR86 genome or RNA replicon (e.g., self-replicating RNA) is derived from a SINV strain Girdwood. In some embodiments, at least one heterologous nsP or portion thereof of the modified SINV-AR86 genome or RNA replicon is derived from nsP2 of a SINV strain Girdwood. In some embodiments, the structural proteins nsP1, nsP3, and nsP4 are from SINV strain AR86, while nsP2 is from SINV strain Girdwood. In some embodiments, nsP4 is from AR86 strain, while nsP1, nsP2, and nsP3 are from Girdwood strain. In some embodiments, nsP3 is from AR86 strain, whereas nsP1, nsP2, and nsP4 are from Girdwood strain. In some embodiments, nsP1 are from AR86 strain, and nsP2, nsP3, whereas nsP4 is from Girdwood (see, e.g., FIGS. 1 and 6).

In some embodiments, a substantial portion of the nucleic acid sequence encoding one or more viral structural proteins has been removed. In some embodiments, the modified viral genome or replicon RNA (e.g., self-replicating RNA) is devoid of the entire sequence encoding viral structural proteins, e.g., the modified viral genome or replicon RNA includes no nucleic acid sequence encoding the structural proteins of the viral unmodified genome or replicon RNA. In some embodiments, the modified alphavirus genome or RNA replicon is devoid of at least a portion of the nucleic acid sequence encoding one or more viral structural proteins. In some embodiments, the modified viral genome or RNA replicon is devoid of a substantial portion of the nucleic acid sequence encoding one or more viral structural proteins. In some embodiments, the modified viral genome or RNA replicon comprises no nucleic acid sequence encoding viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full-length nucleic acid sequence. As described above, the present disclosure provides nucleic acid molecules and constructs which are devoid of partial or complete nucleic acid sequences encoding one or more viral structural proteins.

In some embodiments, the nucleic acid constructs of the disclosure include a nucleic acid sequence encoding a modified alphavirus genome or replicon RNA (e.g., self-replicating RNA), wherein a substantial portion of the nucleic acid sequence encoding one or more structural proteins of the modified alphavirus genome or replicon RNA has been removed, e.g., the modified alphavirus genome or replicon RNA does not include at least a portion of the coding sequence for one or more of the alphavirus structural proteins CP, E1, E2, E3, and 6K.

Non-limiting exemplary embodiments of the nucleic acid constructs of the disclosure can include one or more of the following features. In some embodiments, at least a portion of the nucleic acid sequence encoding one or more of the viral structural proteins CP, E1, E2, E3, and 6K of the unmodified viral genome or replicon RNA (e.g., self-replicating RNA) has been removed. In some embodiments, a portion of or the entire sequence encoding CP has been removed. In some embodiments, a portion of or the entire sequence encoding E1 has been removed. In some embodiments, a portion of or the entire sequence encoding E2 has been removed. In some embodiments, a portion of or the entire sequence encoding E3 has been removed. In some embodiments, a portion of or the entire sequence encoding 6K has been removed. In some embodiments, a portion of or the entire sequence encoding a combination of CP, E1, E2, E3, and 6K has been removed. In some embodiments, the entire sequence encoding viral structural proteins has been removed, e.g., the modified viral genome or replicon RNA includes no nucleic acid sequence encoding the structural proteins of the viral unmodified genome or replicon RNA.

Non-limiting exemplary nucleic acid constructs of the present disclosure can comprise a nucleic acid sequence having at least 80%, at least 85%, at least 90%, at least 95%, at least 96%, at least 97%, at least 98%, at least 99%, or 100% sequence identity to any one of nucleic acid sequence selected from the group consisting of SEQ ID NOs: 1-4. In some embodiments, nucleic acid constructs of the present disclosure can comprise a nucleic acid sequence having at least 80%, at least 85%, at least 90%, at least 95%, at least 96%, at least 97%, at least 98%, at least 99%, or 100% sequence identity to any one of nucleic acid sequence selected from the group consisting of SEQ ID NO: 1. In some embodiments, nucleic acid constructs of the present disclosure can comprise a nucleic acid sequence having at least 80%, at least 85%, at least 90%, at least 95%, at least 96%, at least 97%, at least 98%, at least 99%, or 100% sequence identity to any one of nucleic acid sequence selected from the group consisting of SEQ ID NO: 2. In some embodiments, nucleic acid constructs of the present disclosure can comprise a nucleic acid sequence having at least 80%, at least 85%, at least 90%, at least 95%, at least 96%, at least 97%, at least 98%, at least 99%, or 100% sequence identity to any one of nucleic acid sequence selected from the group consisting of SEQ ID NO: 3. In some embodiments, nucleic acid constructs of the present disclosure can comprise a nucleic acid sequence having at least 80%, at least 85%, at least 90%, at least 95%, at least 96%, at least 97%, at least 98%, at least 99%, or 100% sequence identity to any one of nucleic acid sequence selected from the group consisting of SEQ ID NO: 4.

The nucleic acid constructs (e.g., vectors or srRNA constructs) of the disclosure generally have a length of at least about 2 kb. For example, the nucleic acid constructs (e.g., vectors or srRNAs) can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the nucleic acid constructs (e.g., vectors or srRNAs) can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the nucleic acid constructs (e.g., vectors or srRNAs) can have a length of about 6 kb to about 14 kb. In some embodiments, the nucleic acid constructs (e.g., vectors or srRNAs) can have a length of about 6 kb to about 16 kb.

In some embodiments, the nucleic acid constructs of the disclosure further include one or more expression cassettes. In principle, the nucleic acid constructs disclosed herein can generally include any number of expression cassettes. In some embodiments, the nucleic acid constructs disclosed herein can include at least two, at least three, at least four, at least five, or at least six expression cassettes. The skilled artisan will understand that the term “expression cassette” refers to a construct of genetic material that contains coding sequences and enough regulatory information to direct proper transcription and/or translation of the coding sequences in a cell, in vivo and/or ex vivo. The expression cassette can be inserted into a vector for targeting to a desired host cell and/or into a subject. Accordingly, in some embodiments, the term expression cassette can be used interchangeably with the term “expression construct.” In some embodiments, the term “expression cassette” refers to a nucleic acid construct that includes a gene encoding a protein or functional RNA operably linked to regulatory elements such as, for example, a promoter and/or a termination signal, and optionally, any or a combination of other nucleic acid sequences that affect the transcription or translation of the gene.

In some embodiments, at least one of the expression cassettes can include a promoter operably linked to a heterologous nucleic acid sequence. Accordingly, the nucleic acid constructs as provided herein can find use, for example, as an expression vector that, when including a regulatory element (e.g., a promoter) operably linked to a heterologous nucleic acid sequence, can affect expression of the heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, the nucleic acid molecules of the disclosure further include one or more untranslated regions (UTRs). In some embodiments, at least one of the UTRs is a heterologous UTR. In some embodiments, the 5′ UTR sequence of the modified alphavirus genome or RNA replicon (e.g., self-replicating RNA) is a heterologous 5′ UTR sequence, for example, derived from a heterologous source, e.g., from a different strain of the same alphavirus species or from a different alphavirus species, e.g., UTR sequences from Chikungunya virus. In some embodiments, the 3′ UTR sequence of the modified alphavirus genome or RNA replicon is a heterologous 3′ UTR sequence. In some embodiments, both 5′ UTR and 3′ UTR sequences of the modified alphavirus genome or RNA replicon are heterologous UTR sequences. In some embodiments, the heterologous 5′ UTR and/or 3′ UTR sequences can be from Chikungunya virus. In some embodiments, the heterologous 5′ UTR and/or 3′ UTR sequences can be from a Chikungunya strain S27. In some embodiments, the heterologous 5′ UTR and/or 3′ UTR sequences can be from a Chikungunya strain DRDE.

In some embodiments, at least one of expression cassettes includes a coding sequence for a gene of interest (GOI). In some embodiments, the coding sequence of the GOI is redesigned and/or optimized for a desired property, such as increased stability, potency, and expression (e.g., translation efficiency), which in turns can maximize the impact of producing, delivering, and administering biotherapeutics. For example, in some embodiments, the coding sequence of the GOI is optimized for expression at a level higher than the expression level of a reference coding sequence, for example, 20% higher, 30% higher, 40% higher, 50% higher, 60% higher, 70% higher, 80% higher, 90% higher, or 95% higher than a reference coding sequence. In some embodiments, the reference coding sequence is a wild-type non-optimized sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure can also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence of the GOI is optimized for expression at a level higher than the expression level of a reference coding sequence, such as, for example, a coding sequence that has not been codon-optimized in a target host cell through the use of codons optimized for expression. In some embodiments, the codon-optimized sequence of the GOI results in an increased expression level by at least 30%, at least 40%, at least 50%, at least 60%, at least 70%, at least 80%, at least 90%, at least 95%, or at least 100% compared to a reference coding sequence that has not been codon-optimized. In some embodiments, the codon-optimized sequence of the GOI results in an increased expression level by at least 2-fold, at least 3-fold, at least 4-fold, or at least 5-fold compared to a reference coding sequence that has not been codon-optimized.

In some embodiments, the coding sequence of the GOI is optimized for enhanced RNA stability and/or expression. The stability of RNA generally relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. Several methodologies and techniques useful for evaluation of RNA stability are known, including various in silico methodologies and/or empirical stress-testing of storage of replicons (e.g., self-replicating RNAs) with different GOI codon usage, and its effects on replicon potency (e.g., examine dsRNA in cells following transfection) and gene expression. Additional information in this regard can be found in, for example, Wayment-Steele, H. et al. (2021). Cold Spring Harbor Laboratory (doi.org/10.1101/2020.08.22.262931). Further information regarding principles, strategies, and methods for use in enhancing RNA stability can be found at, for example, Leppek K. et al., Combinatorial optimization of mRNA structure, stability, and translation for RNA-based therapeutics. bioRxiv. (Preprint). Mar. 30, 2021. doi: 10.1101/2021.03.29.437587.

The polypeptide encoded by a GOI can generally be any polypeptide, and can be, for example a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the GOI encodes a polypeptide that can be an antibody, an antigen, an immune modulator, an enzyme, a signaling protein, or a cytokine. In some embodiments, the GOI can encode microbial proteins, viral proteins, bacterial proteins, fungal proteins, mammalian proteins, and combinations of any thereof. Non-limiting examples of GOI include interleukins and interacting proteins, such as G-CSF, GM-CSF, IL-1, IL-10, IL-10-like, IL-11, IL-12, IL-13, IL-14, IL-15, IL-16, IL-17, IL-18, IL-18BP, IL-1-like, IL-1RA, IL-1α, IL-1β, IL-2, IL-20, IL-3, IL-4, IL-5, IL-6, IL-6-like, IL-7, IL-9, IL-21, IL-22, IL-33, IL-37, IL-38, LIF, and OSM. Additional suitable GOIs include, but are not limited to, interferons (e.g., IFN-α, IFN-β, IFN-γ), TNFs (e.g., CD154, LT-β, TNF-α, TNF-β, 4-1BBL, APRIL, CD70, CD153, CD178, GITRL, LIGHT, OX40L, TALL-1, TRAIL, TWEAK, and TRANCE), TGF-β (e.g., TGF-β1, TGF-β2, and TGF-β3), hematopoietins (e.g., Epo, Tpo, Flt-3L, SCF, M-CSF, MSP), chemokines and their receptors (e.g., XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1), immunosuppressive gene products and related transcription factors (e.g., PECAM1, FCGR3A, FOS, NFKB1, JUN, HIF1A, PD-L1, mTOR, STAT5B, and STAT4).

Additional GOIs suitable for the compositions and methods of the disclosure include, but are not limited to, immunostimulatory gene products (e.g., CD27/CD70, CD40, CD40L, B7.1, BTLA, MAVS, OX40, OX40L, RIG-I, and STING), drug resistant mutants/variants of genes, such as ABCB1, ABCC1, ABCG2, AKT1, ALK, BAFF, BCR-ABL, BRAF, CCND1, cMET, EGFR, ERBB2, ERBB3, ERK2, ESR1, GRB2, KRAS, MDR1, MRP1, NTRK1, PDC4, P-gp, PI3K, PTEN, RET, ROS1, RSK1, RSK2, SHIP, and STK11. Also suitable for the compositions and methods of the disclosure includes sequence encoding viral proteins, in particular spike proteins, fiber proteins, structural proteins, and attachment proteins.

In some embodiments, the GOI can encode an antibody or antibody variant (e.g. single chain Fv, bi-specifics, camelids, Fab, and HCAb). In some embodiments, the antibody targets surface molecules associated or upregulated with cancers, or surface molecules associated with infectious disease. In some embodiments, the antibody targets surface molecules having immunostimulatory function, or having immunosuppressive function.

In some embodiments, the GOI can encode an enzyme whose deficiency or mutation is associated with diseases or health conditions, such as, for example, agalsidase beta, agalsidase alfa, imiglucerase, taliglucerase alfa, velaglucerase alfa, alglucerase, sebelipase alpha, laronidase, idursulfase, elosulfase alpha, galsulfase, alglucosidase alpha, and CTFR.

In some embodiments, the GOI can encode a polypeptide selected from antigen molecules, biotherapeutic molecules, or combinations of any thereof. In some embodiments, the GOI can encode a polypeptide selected from tumor-associated antigens, tumor-specific antigens, neoantigens, and combinations of any thereof. In some embodiments, the GOI can encode a polypeptide selected from estrogen receptors, intracellular signal transducer enzymes, and human epidermal growth receptors. In some embodiments, the GOI can encode a biotherapeutic polypeptide selected from immunomodulators, modulators of angiogenesis, modulators of extracellular matrix, modulators of metabolism, neurological modulators, and combinations of any thereof. In some embodiments, the GOI can encode a cytokine selected from chemokines, interferons, interleukins, lymphokines, and tumor necrosis factors. In some embodiments, the GOI can encode an interleukins selected from IL-1α, IL-1β, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ and subunits of any thereof. In some embodiments, the GOI can encode a biotherapeutic polypeptide is selected from IL-12A, IL-12B, IL-1RA, and combinations of any thereof.

In some embodiments, the nucleic acid construct of the disclosure may be incorporated within a vector. In some embodiments, the vector of the disclosure may be single-stranded vector, e.g., ssDNA vector or ssRNA vector. In some embodiments, the vector of the disclosure can be double-stranded vector, e.g., dsDNA vector or dsRNA vector. In some embodiments, the vector of the disclosure can be a plasmid. As described in greater detail below, the vector of the disclosure can be produced using recombinant DNA technology, e.g., polymerase chain reaction (PCR) amplification, rolling circle amplification (RCA), molecular cloning, etc., or chemical synthesis. Accordingly, in some embodiments, the vector of the disclosure can be a fully synthetic vector, e.g., fully synthetic ssDNA vector. In some embodiments, the vector of the disclosure can be a fully synthetic dsDNA vector. In some embodiments, the vector of the disclosure can be a product of a PCR reaction. In some embodiments, the vector of the disclosure can be a product of an RCA reaction. In some embodiments, a vector can be a gene delivery vector. In some embodiments, a vector can be used as a gene delivery vehicle to transfer a gene into a cell.

In some embodiments, the polypeptide encoded by the GOI is a recombinant polypeptide. In some embodiments, the GOI encodes an antigenic HA polypeptide of avian influenza A H5N1. In some embodiments, the GOI encodes a protein relevant to oncology such as ESR1, HER2, and HER3 or a portion thereof. In some embodiments, the GOI encodes a cytokine such as IL-1RA or IL-12.

In some embodiments, the nucleic acid constructs of the disclosure include a nucleic acid sequence encoding a modified genome or RNA replicon (e.g., self-replicating RNA) of an alphavirus species having at least 80%, at least 85%, at least 90%, at least 95%, at least 96%, at least 97%, at least 98%, at least 99%, or 100% sequence identity to a nucleic acid sequence selected from the group consisting of SEQ ID NOs: 1-4. In some embodiments, the nucleic acid constructs of the disclosure include a nucleic acid sequence encoding a modified genome or RNA replicon of an alphavirus species having at least 80%, at least 85%, at least 90%, at least 95%, at least 96%, at least 97%, at least 98%, at least 99%, or 100% sequence identity to the nucleic acid sequence of SEQ ID NO: 1. In some embodiments, the nucleic acid constructs of the disclosure include a nucleic acid sequence encoding a modified genome or RNA replicon of an alphavirus species having at least 80%, at least 85%, at least 90%, at least 95%, at least 96%, at least 97%, at least 98%, at least 99%, or 100% sequence identity to the nucleic acid sequence of SEQ ID NO: 2. In some embodiments, the nucleic acid constructs of the disclosure include a nucleic acid sequence encoding a modified genome or RNA replicon of an alphavirus species having at least 80%, at least 85%, at least 90%, at least 95%, at least 96%, at least 97%, at least 98%, at least 99%, or 100% sequence identity to the nucleic acid sequence of SEQ ID NO: 3. In some embodiments, the nucleic acid constructs of the disclosure include a nucleic acid sequence encoding a modified genome or RNA replicon of an alphavirus species having at least 80%, at least 85%, at least 90%, at least 95%, at least 96%, at least 97%, at least 98%, at least 99%, or 100% sequence identity to the nucleic acid sequence of SEQ ID NO: 4.

TABLE 1
Brief description of the sequences in the Sequence Listing.

| SEQ ID NO | Description |
|---|---|
| 1 | DNA sequence encoding an exemplary base alphavirus replicon (e.g. self- |
| | replicating RNA) vector AR86-Girdwood Hybrid 1, without coding sequence for |
| | a GOI |
| 2 | DNA sequence encoding another exemplary base alphavirus replicon (e.g. self- |
| | replicating RNA) vector AR86-Girdwood Hybrid 2, without coding sequence for |
| | a GOI |
| 3 | DNA sequence encoding yet another exemplary base vector AR86-Girdwood |
| | Hybrid 3, without coding sequence for a GOI |
| 4 | DNA sequence encoding another exemplary base alphavirus replicon (e.g. self- |
| | replicating RNA) vector AR86-Girdwood Hybrid 4 without coding sequence for a |
| | GOI |
| 5 | DNA sequence encoding a srRNA vector of Sindbis AR86-Girdwood Hybrid 1 |
| | with coding sequence for an exemplary GOI: hemagglutinin (HA) gene from |
| | influenza |
| 6 | DNA sequence encoding a srRNA vector of Sindbis AR86-Girdwood Hybrid 2 |
| | with coding sequence for an exemplary GOI: hemagglutinin (HA) gene from |
| | influenza |
| 7 | DNA sequence encoding a srRNA vector of Sindbis AR86-Girdwood Hybrid 3 |
| | with coding sequence for an exemplary GOI: hemagglutinin (HA) gene from |
| | influenza |
| 8 | DNA sequence encoding a srRNA vector of Sindbis AR86-Girdwood Hybrid 4 |
| | with coding sequence for an exemplary GOI: hemagglutinin (HA) gene from |
| | influenza |
| 9 | H5N1 HA coding sequence (Derived from Genbank # AY651334) |
| 10 | Forward primer |
| 11 | Reverse primer |
| 12 | bacteriophage T7 RNA polymerase promoter |
| 13 | T7 terminator sequence |

Nucleic acid sequences having a high degree of sequence identity (e.g., at least 80%, at least 85%, at least 90%, at least 95%, at least 96%, at least 97%, at least 98%, at least 99%, or 100%) to a sequence of a modified genome or RNA replicon (e.g. self-replicating RNA) of an alphavirus species of interest can be identified and/or isolated by using the sequences identified herein (e.g., SEQ ID NOS: 1-4) or any others as they are known in the art, by genome sequence analysis, hybridization, and/or PCR with degenerate primers or gene-specific primers from sequences identified in the alphavirus species genome.

The molecular techniques and methods by which these new nucleic acid constructs were assembled and characterized are described more fully in the Examples herein of the present application.

In some embodiments, the nucleic acid molecules are recombinant nucleic acid molecules. As described above, the term recombinant nucleic acid molecule means any nucleic acid molecule (e.g. DNA, RNA), that is, or results, however indirect, from human manipulation. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature; 3) has been engineered using molecular cloning techniques such that it lacks one or more nucleotides with respect to the naturally occurring nucleotide sequence; and/or 4) has been manipulated using molecular cloning techniques such that it has one or more sequence changes or rearrangements with respect to the naturally occurring nucleotide sequence.

In some embodiments, the nucleic acid molecules disclosed herein are produced using recombinant DNA technology (e.g., polymerase chain reaction (PCR) amplification, cloning, etc.) or chemical synthesis. Nucleic acid molecules as disclosed herein include natural nucleic acid molecules and homologs thereof, including, but not limited to, natural allelic variants and modified nucleic acid molecules in which one or more nucleotide residues have been inserted, deleted, and/or substituted, in such a manner that such modifications provide the desired property in effecting a biological activity as described herein.

A nucleic acid molecule, including a variant of a naturally-occurring nucleic acid sequence, can be produced using a number of methods known to those skilled in the art (see, for example, Sambrook et al., In: Molecular Cloning, A Laboratory Manual, 2nd Edition, Cold Spring Harbor Press, Cold Spring Harbor, N.Y. (1989)). The sequence of a nucleic acid molecule can be modified with respect to a naturally-occurring sequence from which it is derived using a variety of techniques including, but not limited to, classic mutagenesis techniques and recombinant DNA techniques, such as but not limited to site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, recombinational cloning, and chemical synthesis, including chemical synthesis of oligonucleotide mixtures and ligation of mixture groups to “build” a mixture of nucleic acid molecules, and combinations thereof. Nucleic acid molecule homologs can be selected from a mixture of modified nucleic acid molecules by screening for the function of the protein or the replicon (e.g., srRNA) encoded by the nucleic acid molecule and/or by hybridization with a wild-type gene or fragment thereof, or by PCR using primers having homology to a target or wild-type nucleic acid molecule or sequence.

### B. Recombinant Cells

As described in greater detail below, one aspect of the present disclosure relates to recombinant cells that have been engineered to include (e.g., express) a nucleic acid construct as described herein. In some embodiments, a nucleic acid construct (e.g., vector or srRNA) of the present disclosure can be introduced into a host cell to produce a recombinant cell containing the nucleic acid construct and/or srRNA construct. For example, the nucleic acid constructs of the present disclosure can be introduced into a host cell to produce a recombinant cell containing the nucleic acid construct. Accordingly, prokaryotic or eukaryotic cells that contain a nucleic acid construct encoding a modified genome or RNA replicon (e.g., self-replicating RNA) of an alphavirus species as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In one aspect, some embodiments of the disclosure relate to recombinant cells, for example, recombinant eukaryotic cells, e.g., insect cells or animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome-editing techniques such as using guide RNA directed CRISPR/Cas9 or TALEN genome editing. In some embodiments, the nucleic acid construct present in the recombinant host cell as a mini-circle expression vector for a stable or transient expression.

Host cells can be either untransformed cells or cells that have already been transfected with at least one nucleic acid molecule. Accordingly, in some embodiments, host cells can be genetically engineered (e.g., transduced or transformed or transfected) with at least one nucleic acid molecule.

Suitable host cells for cloning or expression of the protein of interest as described herein include prokaryotic or eukaryotic cells described herein. Accordingly, in some embodiments, the recombinant cell is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo, for example, a recombinant cell in a living body, e.g., cell of a transgenic subject. In some embodiments, the subject is a vertebrate animal or an invertebrate animal. In some embodiments, the subject is an insect. In some embodiments, the subject is a mammalian subject. In some embodiments, the mammalian subject is a human subject. In some embodiments, the cell is ex vivo, e.g., has been extracted, as an individual cell or as part of an organ or tissue, from a living body or organism for a treatment or procedure, and then returned to the living body or organism. In some embodiments, the cell is in vitro, e.g., is obtained from a repository. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell.

For expression of glycosylated proteins, suitable host cells can be derived from multicellular organisms (invertebrates and vertebrates). Examples of invertebrate cells include insect cells. Vertebrate cells can also be used as hosts. In this regard, mammalian cell lines that are adapted to grow in suspension can be useful. In some embodiments, the recombinant cell is an animal cell. In some embodiments, the animal cell is a vertebrate animal cell or an invertebrate animal cell. In some embodiments, the recombinant cell is a mammalian cell. In some embodiments, the animal cell is a human cell. In some embodiments, the animal cell is a non-human animal cell. In some embodiments, the cell is a non-human primate cell. Additional examples of useful mammalian host cell lines include monkey kidney CV1 cells transformed by SV40 (COS-7), human embryonic kidney cells (e.g., HEK 293 or HEK 293 cell), baby hamster kidney cells (BHK), mouse sertoli cells (e.g., TM4 cells), human cervical carcinoma cells (HeLa), canine kidney cells (MDCK), buffalo rat liver cells (BRL 3A), human lung cells (W138), human liver cells (Hep G2), mouse mammary tumor (MMT 060562), TRI cells, FS4 cells, Chinese hamster ovary cells (CHO cell), African green monkey kidney cells (Vero cells), human A549 cells, human cervix cells, human CHME5 cells, human PER.C6 cells, NS0 murine myeloma cells, human epidermoid larynx cells, human fibroblast cells, human HUH-7 cells, human MRC-5 cells, human muscle cells, human endothelial cells, human astrocyte cells, human macrophage cells, human RAW 264.7 cells, mouse 3T3 cells, mouse L929 cells, mouse connective tissue cells, mouse muscle cells, and rabbit kidney cells.

In some embodiments, the recombinant cell is selected from the group consisting of African green monkey kidney cell (Vero cell), baby hamster kidney (BHK) cell, Chinese hamster ovary cell (CHO cell), human A549 cell, human cervix cell, human CHME5 cell, human epidermoid larynx cell, human fibroblast cell, human HEK-293 cell, human HeLa cell, human HepG2 cell, human HUH-7 cell, human MRC-5 cell, human muscle cell, mouse 3T3 cell, mouse connective tissue cell, mouse muscle cell, and rabbit kidney cell. In some embodiments, the recombinant cell is a BHK cell. In some embodiments, the BHK cell is a BHK-21 cell. In some embodiments, the recombinant cell is a Vero cell.

In some embodiments, the recombinant cell is an insect cell, e.g., cell of an insect cell line. In some embodiments, the recombinant cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos.55, Sua1B, 4a-3B, Mos.43, MSQ43, and LSB-AA695BB. In some embodiments, the mosquito cell is a cell of a C6/26 cell line.

### C. Cell Culture

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### D. Transgenic Animals

Also provided, in another aspect, are transgenic animals including a nucleic acid construct as described herein (e.g., vector or srRNA molecule). In some embodiments, the transgenic animal is a vertebrate animal or an invertebrate animal. In some embodiments, the transgenic animal is a mammal. In some embodiments, the transgenic mammal is a non-human mammal. Generally, transgenic animals of the present disclosure can be any non-human animal known in the art. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Additional examples of non-human animals suitable for the compositions and methods of the disclosure can include, without limitation, laboratory animals (e.g., mice, rats, hamsters, gerbils, guinea pigs, etc.), livestock (e.g., horses, cattle, pigs, sheep, goats, ducks, geese, chickens, etc.), non-human primates (e.g., apes, chimpanzees, orangutans, monkeys, etc.), fish, amphibians (e.g., frogs, salamanders, etc.), reptiles (e.g., snakes, lizards, etc.), and other animals (e.g., foxes, weasels, rabbits, mink, beavers, ermines, otters, sable, seals, coyotes, chinchillas, deer, muskrats, possums, etc.).

In some embodiments, the transgenic animal is an insect. In some embodiments, the insect is a mosquito. In some embodiments, the transgenic animals of the present disclosure are chimeric transgenic animals. In some embodiments, the transgenic animals of the present disclosure are transgenic animals with germ cells and somatic cells containing one or more (e.g., one or more, two or more, three or more, four or more, etc.) nucleic acid constructs of the present disclosure. In some embodiments, the one or more nucleic acid constructs are stably integrated into the genome of the transgenic animals. In some embodiments, the genomes of the transgenic animals of the present disclosure can comprise any of 1, 2, 3, 4, 5, 6, 7, 8, 9, 10, 11, 12, 13, 14, 15 or more copies of the one or more nucleic acid constructs of the present disclosure.

Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298. In some embodiments, the transgenic non-human host animals of the disclosure are prepared using standard methods known in the art for introducing exogenous nucleic acid into the genome of a non-human animal. In some embodiments, the transgenic animals of the disclosure can be generated using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the transgenic animals of the disclosure can be made using transgenic microinjection technology and do not require the use of homologous recombination technology and thus are considered to be easier to prepare and select than approaches using homologous recombination. In some embodiments, the transgenic animal produces a protein of interest as described herein.

### E. Pharmaceutical Compositions

The nucleic acid constructs, recombinant cells, recombinant polypeptides of the disclosure can be incorporated into compositions, including pharmaceutical compositions. Such compositions generally include one or more of the nucleic acid constructs, recombinant cells, recombinant polypeptides described and provided herein, and a pharmaceutically acceptable excipient, e.g., carrier. In some embodiments, the compositions of the disclosure are formulated for the prevention, treatment, or management of a health condition such as an immune disease or a microbial infection. For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present application are formulated for use as an adjuvant.

Accordingly, in one aspect, provided herein are pharmaceutical compositions including a pharmaceutically acceptable excipient and: a) a nucleic acid construct (e.g., a vector or a srRNA molecule) of the disclosure; b) a recombinant cell of the disclosure; and/or c) a recombinant polypeptide of the disclosure. Non-limiting exemplary embodiments of the pharmaceutical compositions of the disclosure can include one or more of the following features. In some embodiments, provided herein are compositions including a nucleic acid construct (e.g., a vector or a srRNA molecule) as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient.

In some embodiments, the nucleic acid constructs of the disclosure (e.g., a vectors or srRNA molecules) can be used in a naked form or formulated with a delivery vehicle. Exemplary delivery vehicles suitable for the compositions and methods of the disclosure include, but are not limited to liposomes (e.g., neutral or anionic liposomes), microspheres, immune stimulating complexes (ISCOMS), lipid-based nanoparticles (LNP), solid lipid nanoparticles (SLN), polyplexes, polymer nanoparticles, viral replicon particles (VRPs), or conjugated with bioactive ligands, which can facilitate delivery and/or enhance the immune response. These compounds are readily available to one skilled in the art; for example, see Liposomes: A Practical Approach, RCP New Ed, TRL press (1990). Adjuvants other than liposomes and the like are also used and are known in the art. Adjuvants may protect the antigen (e.g., nucleic acid constructs, vectors, srRNA molecules) from rapid dispersal by sequestering it in a local deposit, or they may contain substances that stimulate the host to secrete factors that are chemotactic for macrophages and other components of the immune system. An appropriate selection can be made by those skilled in the art, for example, from those described below.

In some embodiments, a composition of the disclosure can include one or more of the following: physiologic buffer, a liposome, a lipid-based nanoparticle (LNP), a solid lipid nanoparticle (SLN), a polyplex, a polymer nanoparticle, a viral replicon particle (VRP), a microsphere, an immune stimulating complex (ISCOM), a conjugate of bioactive ligand, or a combination of any thereof. In some embodiments, the compositions of the disclosure that formulated in a liposome. In some embodiments, the compositions of the disclosure that formulated in a lipid-based nanoparticle (LNP). LNPs are generally less immunogenic than viral particles. While many humans have preexisting immunity to viral particles there is no pre-existing immunity to LNP. In addition, adaptive immune response against LNP is unlikely to occur which enables repeat dosing of LNP.

The lipids suitable for the compositions and methods described herein can be cationic lipids, ionizable cationic lipids, anionic lipids, or neutral lipids.

In some embodiments, the LNP of the disclosure can include one or more ionizable lipids. As used herein, the term “ionizable lipid” refers to a lipid that is cationic or becomes ionizable (protonated) as the pH is lowered below the pKa of the ionizable group of the lipid, but is more neutral at higher pH values. At pH values below the pKa, the lipid is then able to associate with negatively charged nucleic acids (e.g., oligonucleotides). As used herein, the term “ionizable lipid” includes lipids that assume a positive charge on pH decrease from physiological pH, and any of a number of lipid species that carry a net positive charge at a selective pH, such as physiological pH. Permanently cationic lipids such as DOTMA have proven too toxic for clinical use. The ionizable lipid can be present in lipid formulations according to other embodiments, preferably in a ratio of about 30 to about 70 Mol %, in some embodiments, about 30 Mol %, in other embodiments, about 40 Mol %, in other embodiments, about 45 Mol % in other embodiments, about 47.5 Mol % in other embodiments, about 50 Mol %, in still other embodiments, and about 60 Mol % in yet others (“Mol %” means the percentage of the total moles that is of a particular component). The term “about” in this paragraph signifies a plus or minus range of 5 Mol %. DODMA, or 1,2-dioleyloxy-3-dimethylaminopropane, is an ionizable lipid, as is DLin-MC3-DMA or 0-(Z,Z,Z,Z-heptatriaconta-6,9,26,29-tetraen-19-yl)-4-(N,N-dimethylamino) (“MC3”).

Exemplary ionizable lipids suitable for the compositions and methods of the disclosure includes those described in PCT publications WO2020252589A1 and WO2021000041A1, U.S. Pat. Nos. 8,450,298 and 10,844,028, and Love K. T. et al., Proc Natl Acad Sci USA, Feb. 2, 2010 107 (5) 1864-1869, all of which are hereby incorporated by reference in their entirety. Accordingly, in some embodiments, the LNP of the disclosure includes one or more lipid compounds described in Love K. T. et al. (2010 supra), such as C16-96, C14-110, and C12-200. In some embodiments, the LNP includes an ionizable cationic lipid selected from the group consisting of ALC-0315, C12-200, LN16, MC3, MD1, SM-102, and a combination of any thereof. In some embodiments, the LNP of the disclosure includes C12-200 lipid. The structure of C12-200 lipid is known in the art and described in, e.g., U.S. Pat. Nos. 8,450,298 and 10,844,028, which are hereby incorporated by reference in their entirety. In some embodiments, the C12-200 is combined with cholesterol, C14-PEG2000, and DOPE. In some embodiments, the C12-200 is combined with DSPC and DMG-PEG2000.

In some embodiments, the LNP of the disclosure includes one or more cationic lipids. Several different ionizable cationic lipids have been developed for use in LNP. Suitable cationic lipids include, but are not limited to, 98N12-5, C12-200, C14-PEG2000, DLin-KC2-DMA (KC2), DLin-MC3-DMA (MC3), XTC, MD1, and 7C1. In one type of LNP, a GalNAc moiety is attached to the outside of the LNP and acts as a ligand for uptake into the liver via the asialoglycoprotein receptor. Any of these cationic lipids can be used to formulate LNP for delivery of the srRNA constructs and nucleic acid constructs of the disclosure.

In some embodiments, the LNP of the disclosure includes one or more neutral lipids. Non-limiting neutral lipids suitable for the compositions and methods of the disclosure include DPSC, DPPC, POPC, DOPE, and SM. In some embodiments, the LNP of the disclosure includes one or more ionizable lipid compounds described in PCT publications WO2020252589A1 and WO2021000041A1.

A number of other lipids or combination of lipids that are known in the art can be used to produce a LNP. Non-limiting examples of lipids suitable for use to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Additional non-limiting examples of cationic lipids include 98N12-5, C12-200, C14-PEG2000, DLin-KC2-DMA (KC2), DLin-MC3-DMA (MC3), XTC, MD1, 7C1, and a combination of any thereof. Additional non-limiting examples of neutral lipids include DPSC, DPPC, POPC, DOPE, and SM. Non-limiting examples of PEG-modified lipids include PEG-DMG, PEG-CerC14, and PEG-CerC20.

In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In some embodiments, the compositions of the disclosure that formulated in a polymer nanoparticle. In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant.

In some embodiments, the immunogenic compositions are substantially non-immunogenic to a subject, e.g., compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intraperitoneal administration, intramuscular administration, intranodal administration, intratumoral administration, intraarticular administration, intravenous administration, subcutaneous administration, intravaginal administration, intraocular, rectal, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In these cases, the composition should be sterile and should be fluid to the extent that easy syringeability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the pharmaceutical compositions of the disclosure are formulated for inhalation, such as an aerosol, spray, mist, liquid, or powder. Administration by inhalation may be in the form of either dry powders or aerosol formulations, which are inhaled by a subject (e.g., a patient) either through use of an inhalation device, e.g., a microspray, a pressurized metered dose inhaler, or nebulizer.

In some embodiments, the composition is formulated for one or more of intranasal administration, transdermal administration, intramuscular administration, intranodal administration, intratumoral administration, intraarticular administration, intravenous administration, intraperitoneal administration, oral administration, intravaginal, intraocular, rectal, or intra-cranial administration. In some embodiments, the administered composition results in an increased production of interferon in the subject.

### METHODS OF THE DISCLOSURE

As described in greater detail below, one aspect of the present disclosure relates to, inter alia, methods of functionalizing alphaviruses by replacement of at least a portion of nsP-encoding sequence, methods of producing polypeptide of interest encoded by a gene of interest (GOI), methods of eliciting an immune response in a subject in need thereof, and methods of preventing and/or treating a health condition in a subject in need thereof.

### Methods of Functionalizing an Alphavirus

As outlined above, an aspect of the disclosure relates to a method for functionalizing/engineering an alphavirus genome or RNA replicon (e.g., self-replicating RNA). The methods includes (a) providing a non-functional alphavirus genome or RNA replicon; (b) replacing a nonstructural protein (nsP), or a portion thereof, of the non-functional alphavirus genome or RNA replicon with a heterologous coding sequence for the corresponding nsP or portion thereof derived from a different alphavirus strain to generate a modified alphavirus genome or RNA replicon; (c) assessing functionality of the modified alphavirus genome or RNA replicon; (d) identifying the modified alphavirus genome or RNA replicon as being functional if the modified alphavirus genome or RNA replicon is capable of RNA replication and/or expression.

In some embodiments, the heterologous nsP or portion thereof is derived from another strain of the same alphavirus species. In some embodiments, the heterologous nsP or portion thereof is derived from another alphavirus species. In some embodiments, the heterologous nsP or portion thereof is nsP1, nsP2, nsP3, nsP4, or a portion of any thereof. In some embodiments, the non-functionality of the alphavirus genome or RNA replicon (e.g., self-replicating RNA) is determined by a deficiency in self-replication within a host cell. In general, functionality of the modified alphavirus genome or RNA replicons of the disclosure can be evaluated by using one or more assays and methodologies known in the art, such as detection of RNA replication, detection of viral protein expression, detection of cytopathic effect (CPE), and detection of heterologous transgene expression. In particular, a non-functional alphavirus can be identified as being incapable of self-replication within a cell culture or primary cell line, for example, but not limited to, BHK, VERO, or HEK293. As described above, non-functionality of an alphavirus can be determined when the deposited alphavirus sequence (e.g., sequences retrieved from public databases) is found insufficient when reproduced synthetically to self-replicate.

### Methods of Producing a Polypeptide of Interest

In an aspect, provided herein are methods for producing a polypeptide of interest, wherein the methods include culturing a recombinant cell including a nucleic acid construct as disclosed herein under conditions wherein the recombinant cell produces the polypeptide encoded by the GOI. In another aspect, provided herein are methods for producing a polypeptide of interest in a subject, wherein the methods include administering to the subject a nucleic acid construct as disclosed herein. In some embodiments, the subject is vertebrate animal or an invertebrate animal. In some embodiments, the subject is a mammalian subject. In some embodiments, the mammalian subject is a human subject. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life. In some embodiments, the N-terminus of the produced polypeptide can be further chemically or enzymatically modified to increase half-life. In some embodiments, the C-terminus of the produced polypeptide is chemically and/or enzymatically modified to increase half-life. Non-limiting examples of chemical and enzymatic modifications suitable for the methods described herein include PEGylation, XTENylation, PASylation®, ELPylation, and HAPylation. Techniques, systems, and reagents suitable for these modifications are known in the art. According, in some embodiments, the polypeptide produced by the methods described herein can be PEGylated, XTENylated, PASylated, ELPylated, and/or HAPylated to increase half-life. In some embodiments the produced polypeptide is conjugated to another protein or peptide (e.g., serum albumin, an antibody Fc domain, transferrin, GLK, or CTP peptide) to increase half-life.

In an embodiment, methods for producing a polypeptide of interest comprises (i) rearing a transgenic animal of the present disclosure, or (ii) culturing a recombinant cell comprising a nucleic acid construct of the present disclosure under conditions wherein the recombinant cell produces the polypeptide encoded by the GOI.

In an embodiment, methods for producing a polypeptide of interest in a subject comprises administering to the subject a nucleic acid construct of the present disclosure. In some embodiments, the subject is vertebrate animal or an invertebrate animal. In some embodiments, the animal is an insect. In some embodiments, the subject is a mammalian subject. In some embodiments, the mammalian subject is a human subject.

### Methods of Inducing Pharmacodynamic Effect, Eliciting Immune Response, Preventing, or Treating Health Conditions

Administration of any one of the therapeutic compositions described herein, e.g., nucleic acid constructs (e.g., vectors or srRNA molecules), recombinant cells, recombinant polypeptides, and/or pharmaceutical compositions, can be used in the treatment of relevant health conditions, such as proliferative disorders (e.g., cancers), infectious diseases (e.g., acute infections, chronic infections, or viral infections), rare diseases, and/or autoimmune diseases, and/or inflammatory diseases. In some embodiments, the nucleic acid constructs (e.g., vectors or srRNA molecules), recombinant cells, recombinant polypeptides, and/or pharmaceutical compositions as described herein can be incorporated into therapeutic agents for use in methods of treating an individual who has, who is suspected of having, or who may be at high risk for developing one or more relevant health conditions or diseases. Exemplary health conditions or diseases can include, without limitation, cancers, immune diseases, gene therapy, gene replacement, cardiovascular diseases, age-related pathologies, acute infection, and chronic infection. In some embodiments, the individual is a patient under the care of a physician.

In some embodiments, the nucleic acid constructs (e.g., vectors or srRNA molecules), recombinant cells, recombinant polypeptides, and/or pharmaceutical compositions as described herein can be useful for inducing a pharmacodynamic effect in a subject. In some embodiments, the nucleic acid constructs (e.g., vectors or srRNA molecules), recombinant cells, recombinant polypeptides, and/or pharmaceutical compositions as described herein can be useful for modulating, e.g., eliciting or suppressing, an immune response in a subject in need thereof. Accordingly, in one aspect, provided herein are methods for eliciting an immune response in a subject in need thereof, the method includes administering to the subject a composition including: a) a nucleic acid construct of the disclosure (e.g., vector or srRNA molecule); b) a recombinant cell of the disclosure; c) a recombinant polypeptide of the disclosure; and/or d) a pharmaceutical composition of the disclosure.

The analysis of the compositions described herein for their capacity to confer a pharmacodynamic effect can be carried out in vivo and/or ex vivo. Examples of pharmacodynamic effects that can be analyzed include: immunogenicity effect (e.g., eliciting an immune response in vivo), a biomarker response, a therapeutic effect, a prophylactic effect, a desired effect, an undesired effect, an adverse effect, and effect in a disease model. In some embodiments, the assessment of pharmacodynamic effects includes assessing induction of an immune response in vivo. In some embodiments, the assessment of pharmacodynamic effects includes assessing induction of cytokine pathways that can potentiate an immune response and prevent angiogenesis and metastasis.

In another aspect, provided herein are methods for preventing and/or treating a health condition in a subject in need thereof, the method includes prophylactically or therapeutically administering to the subject a composition including: a) a nucleic acid construct of the disclosure (e.g., vector or srRNA molecule); b) a recombinant cell of the disclosure; c) a recombinant polypeptide of the disclosure; and/or d) a pharmaceutical composition of any one of the disclosure.

In some embodiments, the health condition is a proliferative disorder or a microbial infection. In some embodiments, the subject has or is suspected of having a condition associated with proliferative disorder or a microbial infection.

In some embodiments, the disclosed composition is formulated to be compatible with its intended route of administration. For example, the nucleic acid constructs, recombinant cells, recombinant polypeptides, and/or pharmaceutical compositions of the disclosure can be given orally or by inhalation, but it is more likely that they will be administered through a parenteral route. Examples of parenteral routes of administration include, for example, intravenous, intranodal, intradermal, subcutaneous, transdermal (topical), transmucosal, intravaginal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject nucleic acid constructs, recombinant cells, recombinant polypeptides, and/or pharmaceutical compositions of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects can be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma can be measured, for example, by high performance liquid chromatography.

The therapeutic compositions described herein, e.g., nucleic acid constructs, recombinant cells, recombinant polypeptides, and/or pharmaceutical compositions, can be administered one from one or more times per day to one or more times per week; including once every other day. The skilled artisan will appreciate that certain factors can influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to nucleic acid constructs and recombinant polypeptides, the therapeutically effective amount of a nucleic acid construct or recombinant polypeptide of the disclosure (e.g., an effective dosage) depends on the nucleic acid construct or recombinant polypeptide selected. For instance, single dose amounts in the range of approximately 0.001 to 0.1 mg/kg of patient body weight can be administered. In some embodiments, about 0.005, 0.01, 0.05 mg/kg can be administered. In some embodiments, single dose amounts in the range of approximately 0.03 μg to 300 μg/kg of patient body weight can be administered. In some embodiments, single dose amounts in the range of approximately 0.3 mg to 3 mg/kg of patient body weight can be administered.

As discussed supra, a therapeutically effective amount includes an amount of a therapeutic composition that is sufficient to promote a particular effect when administered to a subject, such as one who has, is suspected of having, or is at risk for a health condition, e.g., a disease or infection. In some embodiments, an effective amount includes an amount sufficient to prevent or delay the development of a symptom of the disease or infection, alter the course of a symptom of the disease or infection (for example but not limited to, slow the progression of a symptom of the disease or infection), or reverse a symptom of the disease or infection. It is understood that for any given case, an appropriate effective amount can be determined by one of ordinary skill in the art using routine experimentation.

The efficacy of a treatment including a disclosed therapeutic composition for the treatment of disease or infection can be determined by the skilled clinician. However, a treatment is considered effective treatment if at least any one or all of the signs or symptoms of disease or infection are improved or ameliorated. Efficacy can also be measured by failure of an individual to worsen as assessed by hospitalization or need for medical interventions (e.g., progression of the disease or infection is halted or at least slowed). Methods of measuring these indicators are known to those of skill in the art and/or described herein. Treatment includes any treatment of a disease or infection in a subject or an animal (some non-limiting examples include a human, or a mammal) and includes: (1) inhibiting the disease or infection, e.g., arresting, or slowing the progression of symptoms; or (2) relieving the disease or infection, e.g., causing regression of symptoms; and (3) preventing or reducing the likelihood of the development of symptoms.

In some embodiments, the nucleic acid constructs (e.g., vectors or srRNA molecules), recombinant cells, recombinant polypeptides, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject, for example, by at least 30%, at least 40%, at least 50%, at least 60%, at least 70%, at least 80%, at least 90%, at least 95%, or at least 100% as compared to interferon production in a subject that has not been administered with the composition. In some embodiments, the administered composition results in an increased production of interferon in the subject. In some embodiments of the disclosed methods, the subject is a mammal. In some embodiments, the mammal is human.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringeability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, asorbic acid, thimerosal, and the like.

Sterile injectable solutions can be prepared by incorporating the nucleic acid constructs (e.g., vectors or srRNA molecules), recombinant cells, and/or recombinant polypeptides in the required mount in an appropriate solvent with one or a combination of ingredients enumerated above, as required, followed by filtered sterilization.

When the nucleic acid constructs (e.g., vectors or srRNA molecules), recombinant cells, recombinant polypeptides, and/or pharmaceutical compositions are suitably protected, as described above, they can be orally administered, for example, with an inert diluent or an assimilable edible carrier. The nucleic acid constructs (e.g., vectors or srRNA molecules), recombinant cells, recombinant polypeptides, and/or pharmaceutical compositions and other ingredients can also be enclosed in a hard or soft shell gelatin capsule, compressed into tablets, or incorporated directly into the individual's diet. For oral therapeutic administration, the active compound can be incorporated with excipients and used in the form of ingestible tablets, buccal tablets, troches, capsules, elixirs, suspensions, syrups, wafers, and the like.

In some embodiments, the nucleic acid constructs (e.g., vectors or srRNA molecules) and recombinant polypeptides of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

As discussed supra, several different ionizable cationic lipids have been developed for use in LNP. These include C12-200, MC3, LN16, and MID among others. For example, in one type of LNP, a GalNAc moiety is attached to the outside of the LNP and acts as a ligand for uptake in to the liver via the asialoglycoprotein receptor. Any of these cationic lipids can be used to formulate LNP for delivery of the nucleic acid constructs and recombinant polypeptides of the disclosure to the liver.

In some embodiments, a LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

As described supra, a number of lipids or combination of lipids that have been developed for use in LNP can be used to produce a LNP of the disclosure. Non-limiting examples of lipids suitable for use in production of LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Non-limiting examples of cationic lipids suitable for use in production of LNPs include 98N12-5, C12-200, DLin-KC2-DMA (KC2), DLin-MC3-DMA (MC3), XTC, MID, and 7C1. Non-limiting examples of neutral lipids suitable for use in production of LNPs include DPSC, DPPC, POPC, DOPE, and SM. Non-limiting examples of PEG-modified lipids suitable for use in production of LNPs include PEG-DMG, PEG-CerC14, and PEG-CeraC20.

In some embodiments, the lipids can be combined in any number of molar ratios to produce a LNP. In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In addition, the polynucleotide(s) can be combined with lipid(s) in a wide range of molar ratios to produce a LNP.

In some embodiments, the therapeutic compositions described herein, e.g., nucleic acid constructs (e.g., vectors or srRNA molecules), recombinant cells, recombinant polypeptides, and/or pharmaceutical compositions are incorporated into therapeutic compositions for use in methods of preventing or treating a subject who has, who is suspected of having, or who can be at high risk for developing one or more relevant health conditions or diseases. Exemplary health conditions or diseases can include, without limitation, cancers, immune diseases, autoimmune diseases, inflammatory diseases, gene therapy, gene replacement, cardiovascular diseases, age-related pathologies, rare disease, acute infection, and chronic infection.

In some embodiments, the nucleic acid constructs (e.g., vectors or srRNA molecules), recombinant cells, recombinant polypeptides, and/or pharmaceutical compositions, can be useful in the treatment and/or prevention of immune diseases, autoimmune diseases, or inflammatory diseases such as, for example, glomerulonephritis, inflammatory bowel disease, nephritis, peritonitis, psoriatic arthritis, osteoarthritis, Still's disease, Familiar Mediterranean Fever, systemic scleroderma and sclerosis, inflammatory bowel disease (IBD), Crohn's disease, ulcerative colitis, acute lung injury, meningitis, encephalitis, uveitis, multiple myeloma, glomerulonephritis, nephritis, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, hemolytic anemia, myasthenia gravis, lupus nephritis, lupus erythematosus, rheumatoid arthritis (RA), ankylosing spondylitis, pemphigus, Graves' disease, Hashimoto's thyroiditis, small vessel vasculitides, Omen's syndrome, chronic renal failure, autoimmune thyroid disease, acute infectious mononucleosis, HIV, herpes virus associated diseases, human virus infections, coronavirus, other enterovirus, herpes virus, influenza virus, parainfluenza virus, respiratory syncytial virus or adenovirus infection, bacteria pneumonia, wounds, sepsis, cerebral stroke/cerebral edema, ischaemia-reperfusion injury, and hepatitis C.

Non-limiting examples of inflammatory suitable for the methods of the disclosure include inflammatory diseases such as asthma, inflammatory bowel disease (IBD), chronic colitis, splenomegaly, and rheumatoid arthritis.

Examples of autoimmune diseases suitable for the methods of the disclosure include, but are not limited to, rheumatoid arthritis, osteoarthritis, Still's disease, Familiar Mediterranean Fever, systemic sclerosis, multiple sclerosis, ankylosing spondylitis, Hashimoto's thyroiditis, systemic lupus erythematosus, Sjogren's syndrome, diabetic retinopathy, diabetic vasculopathy, diabetic neuralgia, insulitis, psoriasis, alopecia areata, warm and cold autoimmune hemolytic anemia (AIHA), pernicious anemia, acute inflammatory diseases, autoimmune adrenalitis, chronic inflammatory demyelinating polyneuropathy (CIDP), Lambert-Eaton syndrome, lichen sclerosis, Lyme disease, Graves disease, Behçet's disease, Meniere's disease, reactive arthritis (Reiter's syndrome), Churg-Strauss syndrome, Cogan syndrome, CREST syndrome, pemphigus vulgaris and pemphigus foliaceus, bullous pemphigoid, polymyalgia rheumatica, polymyositis, primary biliary cirrhosis, pancreatitis, peritonitis, psoriatic arthritis, rheumatic fever, sarcoidosis, Sjörgensen syndrome, scleroderma, celiac disease, stiff-man syndrome, Takayasu arteritis, transient gluten intolerance, autoimmune uveitis, vitiligo, polychondritis, dermatitis herpetiformis (DH) or Duhring's disease, fibromyalgia, Goodpasture syndrome, Guillain-Barre syndrome, Hashimoto thyroiditis, autoimmune hepatitis, inflammatory bowel disease (IBD), Crohn's disease, colitis ulcerosa, myasthenia gravis, immune complex disorders, glomerulonephritis, polyarteritis nodosa, anti-phospholipid syndrome, polyglandular autoimmune syndrome, idiopathic pulmonary fibrosis, idiopathic thrombocytopenic purpura (ITP), urticaria, autoimmune infertility, juvenile rheumatoid arthritis, sarcoidosis, and autoimmune cardiomyopathy.

In some embodiments, the therapeutic compositions described herein, e.g., nucleic acid constructs (e.g., vectors or srRNA molecules), recombinant cells, recombinant polypeptides, and/or pharmaceutical compositions are incorporated into therapeutic compositions for use in methods of preventing or treating a subject who has, who is suspected of having, or who can be at high risk for developing a microbial infection (e.g., bacterial infection, micro-fungal infection, or viral infection). Non-limiting examples of infection suitable for the methods of the disclosure include infections with viruses such as human immunodeficiency virus (HIV), hepatitis B virus (HBV), hepatitis B virus (HCV), Cytomegalovirus (CMV), respiratory syncytial virus (RSV), human papillomavirus (HPV), Epstein-Barr virus (EBV), severe acute respiratory syndrome coronavirus 2 (SARS-CoV2), severe acute respiratory syndrome coronavirus (SARS-CoV), Middle East Respiratory Syndrome (MERS), influenza virus, and Ebola virus. Additional infections suitable for the methods of the disclosure include infections with intracellular parasites such as Leishmania, Rickettsia, Chlamydia, Coxiella, Plasmodium, Brucella, mycobacteria, Listeria, Toxoplasma and Trypanosoma. In some embodiments, the microbial infection is a bacterial infection. In some embodiments, the microbial infection is a fungal infection. In some embodiments, the microbial infection is a viral infection.

In some embodiments, the health condition is a rare disease, e.g., a disease or condition that affects less than 200,000 people in the United States, as defined by The Orphan Drug Act (www.fda.gov/patients/rare-diseases-fda) and/or an inflammatory and/or autoimmune disorder. In some embodiments, the subject has or is suspected of having a condition associated with an inflammatory and/or autoimmune disorder and/or a rare disease (e.g. including but not limited to Familial Mediterranean Fever or adult onset Still's disease).

### Additional Therapies

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein. In particular, some embodiments of the disclosure provide kits for eliciting an immune response in a subject. Some other embodiments relate to kits for the prevention of a health condition in a subject in need thereof. Some other embodiments relate to kits for methods of treating a health condition in a subject in need thereof. For example, provided herein, in some embodiments, are kits that include one or more of the nucleic acid constructs (e.g., vectors or srRNA molecules), recombinant cells, recombinant polypeptides, and/or pharmaceutical compositions as provided and described herein, as well as written instructions for making and using the same.

In some embodiments, the kits of the disclosure further include one or more means useful for the administration of any one of the provided nucleic acid constructs (e.g., vectors or srRNA molecules), recombinant cells, recombinant polypeptides, and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs (e.g., vectors or srRNA molecules), recombinant cells, recombinant polypeptides, and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers, reconstitution solutions, wash buffers, control reagents, control expression vectors, negative controls, positive controls, reagents suitable for in vitro production of the provided nucleic acid constructs (e.g., vectors or srRNA molecules), recombinant cells, recombinant polypeptides, and/or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the nucleic acid constructs (e.g., vectors or srRNA molecules), recombinant cells, recombinant polypeptides, and/or pharmaceutical compositions as provided and described herein in one container (e.g., in a sterile glass or plastic vial) and a further therapeutic agent in another container (e.g., in a sterile glass or plastic vial).

In another embodiment, the kit includes a combination of the compositions described herein, including one or more nucleic acid constructs (e.g., vectors or srRNA molecules), recombinant cells, and/or recombinant polypeptides of the disclosure in combination with one or more additional therapeutic agents formulated together in a pharmaceutical composition and, optionally, in a single, common container.

If the kit includes a pharmaceutical composition for parenteral administration to a subject, the kit can include a device (e.g., an injection device or catheter) for performing such administration. For example, the kit can include one or more hypodermic needles or other injection devices as discussed above containing one or more nucleic acid constructs (e.g., vectors or srRNA molecules), recombinant cells, and/or recombinant polypeptides of the disclosure.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Design and Construction of Base Vectors SINV AR86-Girdwood

This Example describes the results of experiments performed to construct a number of base alphavirus vectors (e.g., without a heterologous gene) that were subsequently used for expression of a gene of interest (e.g., a hemagglutinin (HA) gene from influenza).

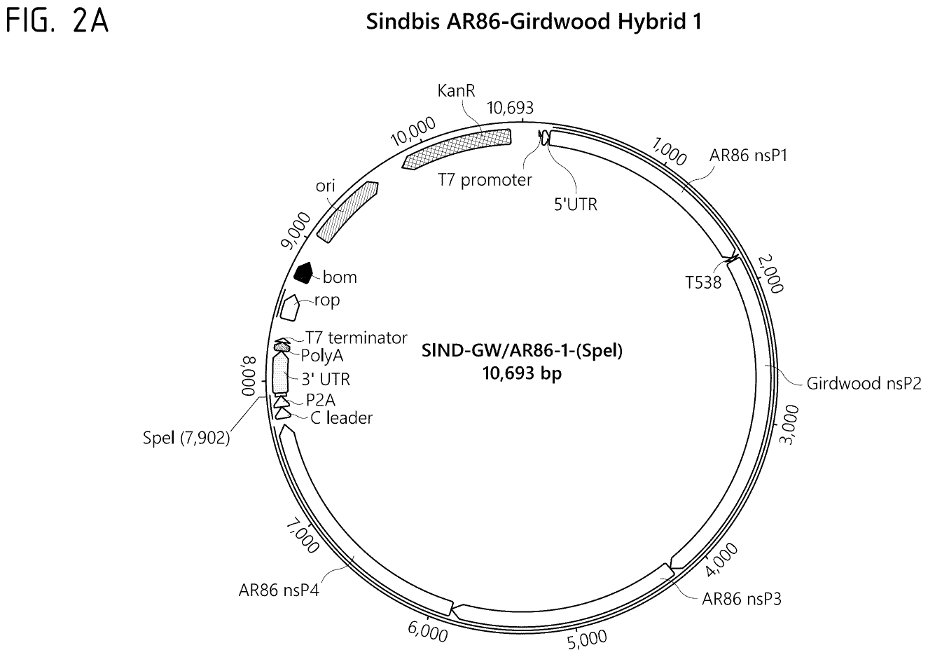

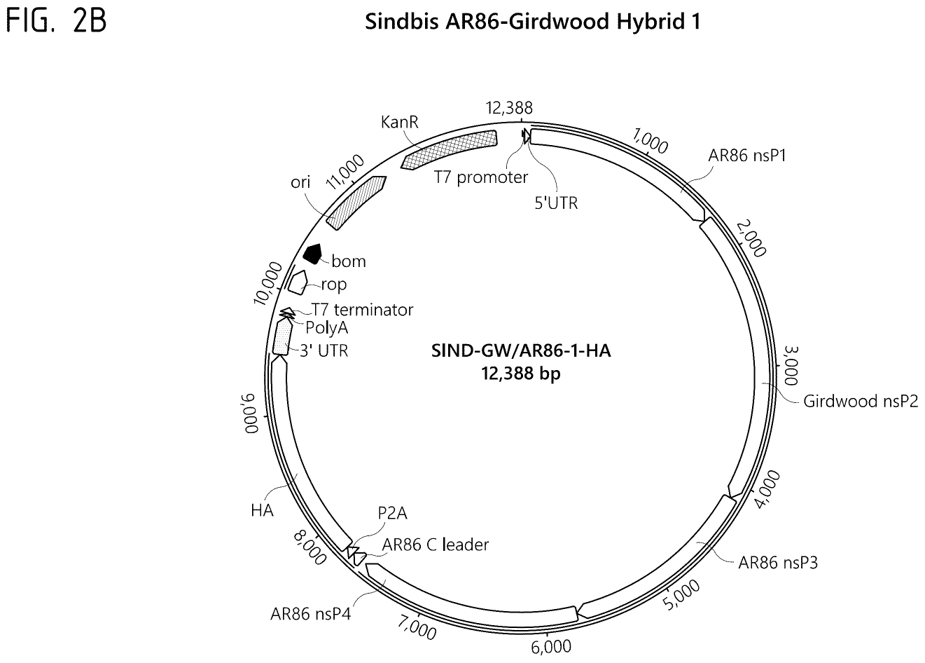

The Sindbis AR86-Girdwood hybrid 1 vector described in FIG. 2A was constructed as follows. The base SINV AR86-Girdwood hybrid 1 vector was synthesized de novo in four ˜4 kb parts (Twist Bioscience) from an AR-86 reference sequence (Genbank U38305) with a unique restriction enzyme cut site (SpeI, 5‘-A’CTAG,T-3′) in place of the coding sequence of the SINV structural genes (where the 5′ A is the next nucleotide after a P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′; SEQ ID NO: 12) was included upstream of the SINV genome sequence, and downstream contained a polyA sequence followed by a unique restriction enzyme site (SapI, 5′-GCTCTTC(N)₁′(N)₃,-3′) followed by a T7 terminator sequence (5′-AACCCCTCTCTAAACGGAGGGGTTTTTTT-3′; SEQ ID NO: 13) followed by a unique restriction enzyme cut site (NotI, 5‘-GC’GGCC,GC-3′). The parts were combined in a five-piece Gibson Assembly® reaction (e.g., a linearized pYL backbone and the four synthesized fragments). In the resulting vector, the AR86 nsP2 gene was replaced with the Girdwood nsP2 gene (Genbank MF459683), resulting in the final SINV AR86-Girdwood hybrid 1 base vector (SEQ ID NO: 1).

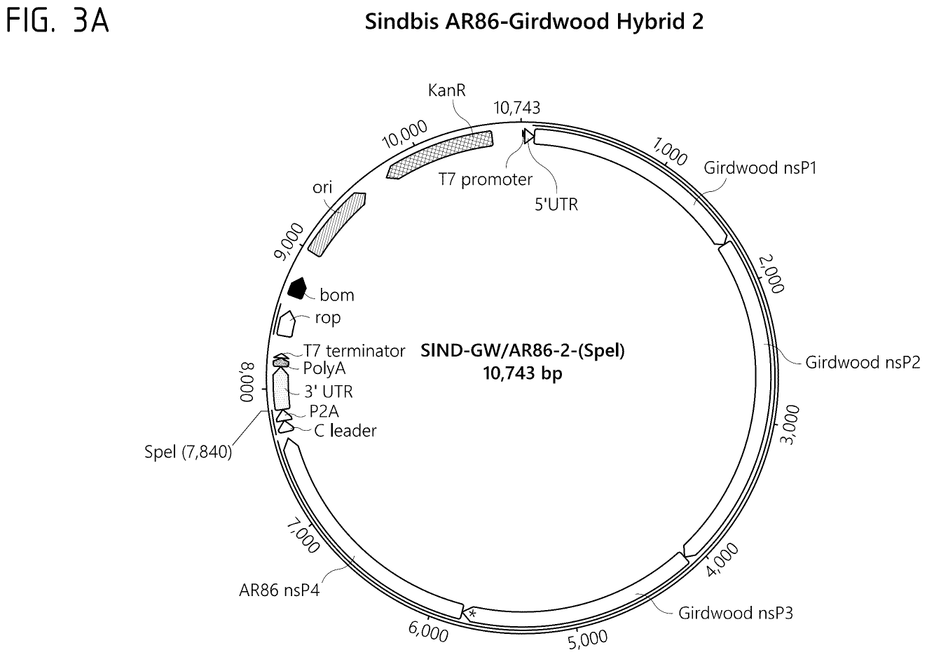

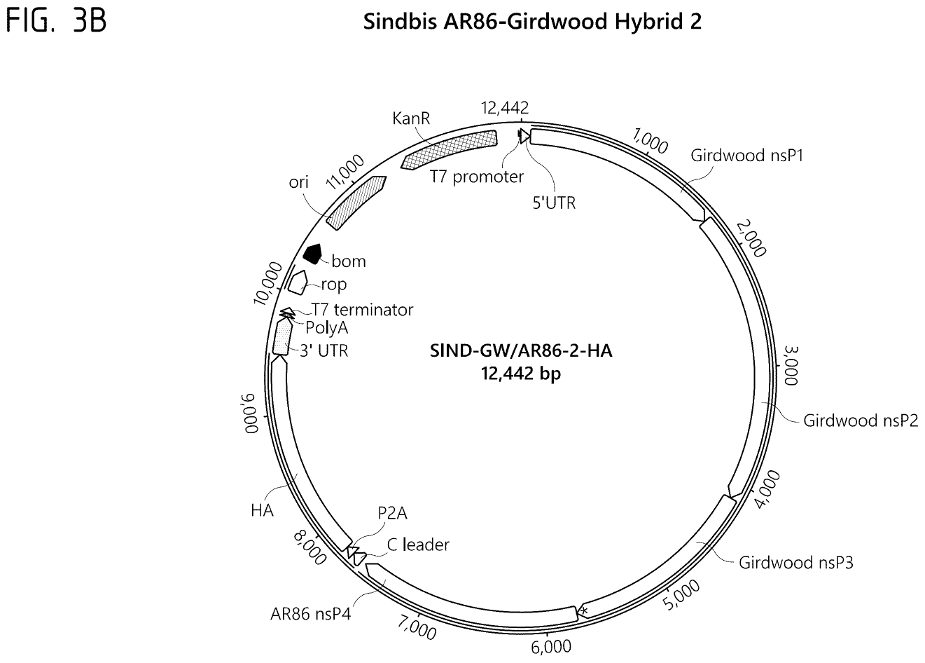

The Sindbis AR86-Girdwood hybrid 2 vector described in FIG. 3A was constructed as follows. The base SINV Girdwood vector was synthesized de novo in four ˜4 kb parts (Twist Bioscience, Thermo Fisher GeneArt) from a Girdwood strain reference sequence (Genbank MF459683) with a unique restriction enzyme cut site (SpeI, 5‘-A’CTAG,T-3′) in place of the coding sequence of the SINV structural genes (where the 5′ A is the next nucleotide after a P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′; SEQ ID NO: 12) was included upstream of the SINV genome sequence, and downstream contained a polyA sequence followed by a unique restriction enzyme site (SapI, 5′-GCTCTTC(N)₁′(N)₃,-3′) followed by a T7 terminator sequence (5′-AACCCCTCTCTAAACGGAGGGGTTTTTTT-3′; SEQ ID NO: 13) followed by a unique restriction enzyme cut site (NotI, 5‘-GC’GGCC,GC-3′). The parts were combined in a five-piece Gibson Assembly® reaction (e.g., a linearized pYL backbone and the four synthesized fragments). In the resulting vector, the Girdwood nsP4 gene was replaced with the AR86 nsP4 gene, resulting in the final SINV AR86-Girdwood hybrid 2 base vector (SEQ ID NO: 2).

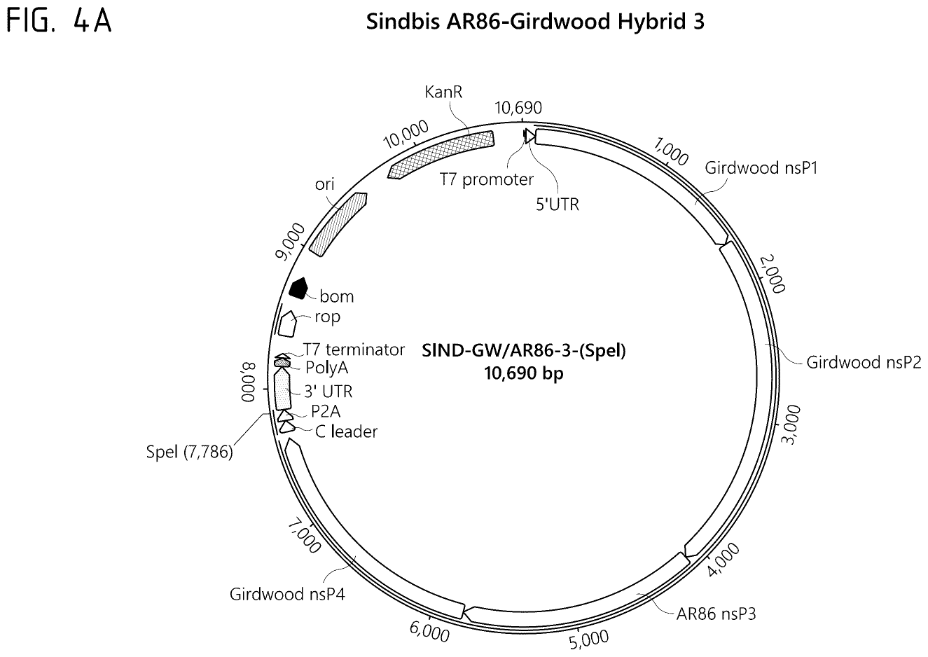

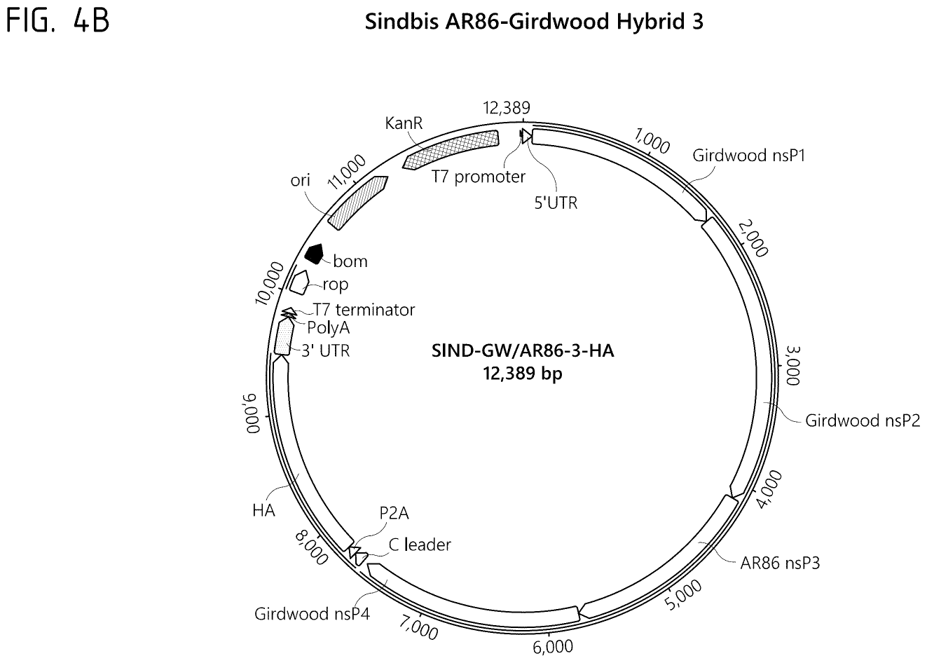

The Sindbis AR86-Girdwood hybrid 3 vector described in FIG. 4A was constructed as follows. Similar to the Sindbis AR86-Girdwood hybrid 2 base vector, the Sindbis AR86-Girdwood hybrid 3 base vector was constructed by instead replacing the Girdwood nsP3 gene with the AR86 nsP3 gene (SEQ ID NO: 3).

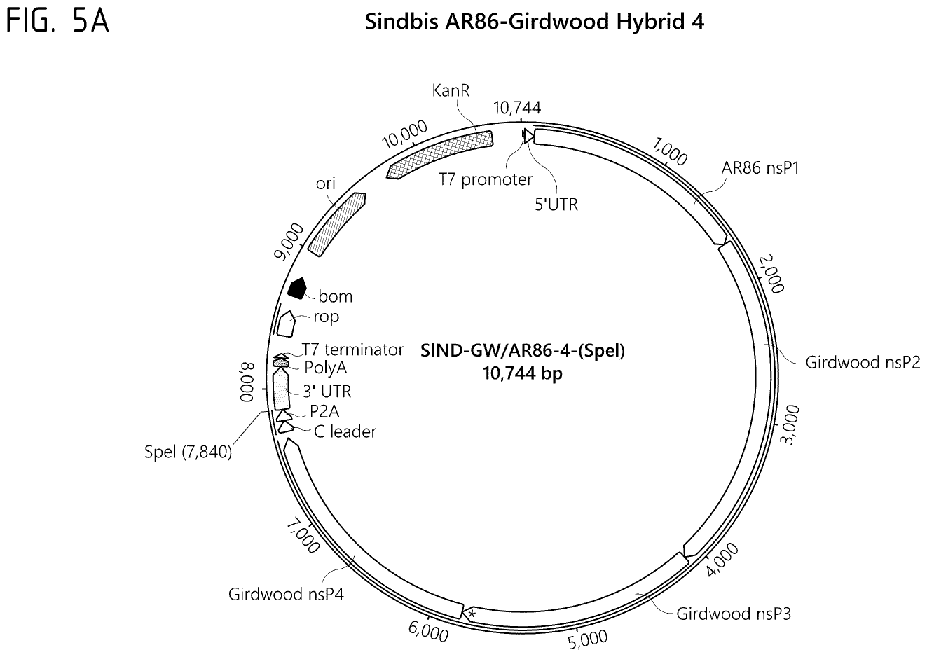

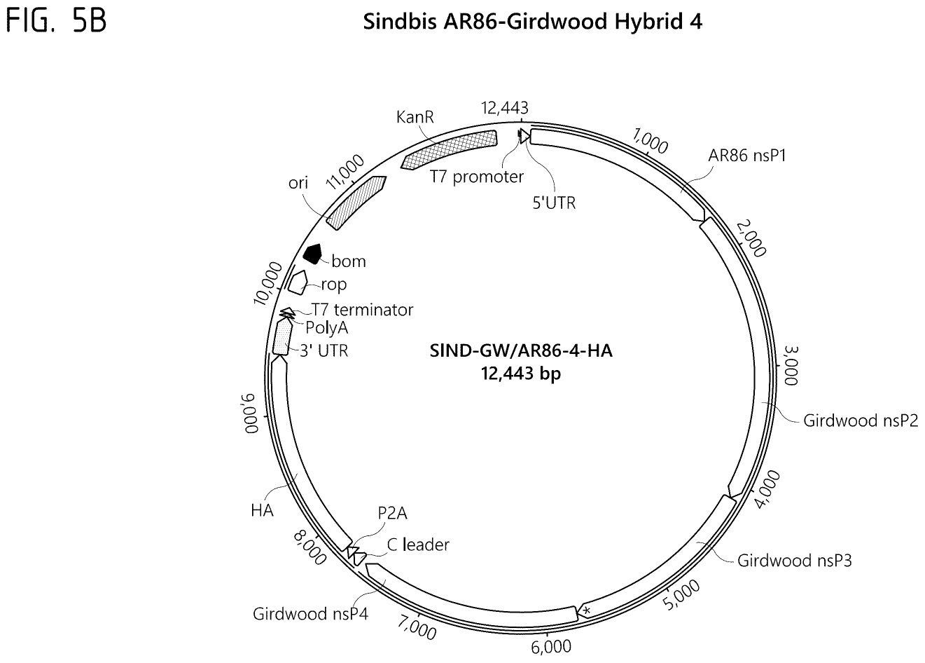

The Sindbis AR86-Girdwood hybrid 4 vector described in FIG. 5A was constructed as follows. Similar to the Sindbis AR86-Girdwood hybrid 2 base vector, the Sindbis AR86-Girdwood hybrid 4 base vector was constructed by instead replacing the Girdwood nsP1 gene with the AR86 nsP1 gene (SEQ ID NO: 4).

### Example 2

### Construction of Modified Alphavirus Vectors for Expression of a Gene of Interest

The alphavirus vectors in FIGS. 2B, 3B, 4B, and 5B were constructed by linearization of the empty base vector in FIGS. 2A, 3A, 4A, and 5A, respectively, by SpeI endonuclease digestion. The hemagglutinin (HA) gene from influenza (Genbank #AY651334) was codon refactored for human expression in silico and synthesized (IDT). The synthetic product was amplified using the following primers which add 30 bp of flanking homology ends to the base vector on the PCR product.

Forward primer:
SEQ ID NO: 10
(5′-GCTGGAGACGTGGAGGAGAACCCTGGACCTATGGAGAAAATAGTGC
TTCTTTTTG-3′;).
Reverse primer:
SEQ ID NO: 11
(5′-GCTGGTCGGGTCATTGGGGCGTAGCGGTCAAATGCAAATTCTGCAT
TGTAACG-3′;),

The digestion product (i.e., linearized vector) and the PCR product were combined by two-fragment Gibson Assembly® reaction to result in the final vectors each containing a H5N1 HA coding sequence placed under control of a 26S subgenomic promoter (SEQ ID NOs: 5-8 for AR86-Girdwood Hybrids 1-4, respectively).

### Example 3

### Improve Functionality of Defective Alphavirus Genome and RNA Replicon

This Example describes the results of experiments performed to demonstrate that a defective (non-functional) RNA replicon (e.g. self-replicating RNA) can be functionalized (e.g., caused to be functional) by replacement of a defective nsP sequence with a corresponding nsP sequence from a functional alphavirus.

In vitro transcription: Self-replicating RNA (srRNA) was prepared by in vitro transcription using a plasmid DNA template linearized by enzymatic digestion. In these examples, the DNA was either linearized with NotI, which cuts downstream of the T7 terminator, or linearized with SapI, which cuts at the end of the poly(A). Bacteriophage T7 polymerase was used for in vitro transcription with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). srRNA was purified using phenol/chloroform extraction, LiCl precipitation, or column purification (Monarch® RNA Cleanup Kit, NEB). srRNA concentration was determined by absorbance at 260 nm (Nanodrop, Thermo Fisher Scientific).

Replication: srRNA was transformed by electroporation into BHK-21 or Vero cells (e.g. 4D-Nucleofector™, Lonza). At 17-20 h following transformation, the cells were fixed and permeabilized (eBioscience™ Foxp3/Transcription Factor Staining Buffer Set, Invitrogen) and stained using a PE-conjugated anti-double stranded RNA (dsRNA) mouse monoclonal antibody (J2, Scicons) to quantify the frequency of dsRNA+ cells and the mean fluorescence intensity (MFI) of dsRNA in individual cells by fluorescence flow cytometry.

A non-functional RNA replicon (e.g. self-replicating RNA) fails to exhibit a signal after staining cells transfected with srRNA (see RNA replicons (e.g. self-replicating RNAs) marked with an X), where transfected functional RNA replicons (e.g. self-replicating RNAs) produce detectible dsRNA (see RNA replicons marked with a “V” symbol) which is indicative of RNA replication and is a necessary for 26S RNA transcription and subsequent transgene expression (FIG. 6). Without being bound to any particular theory, it is believe that functional, replicating srRNAs as described herein can be considered used as practical vectors to induce pharmacokinetic effects.

### Example 4

### In Vitro Evaluation of Modified Alphavirus Vectors

This Example describes the results of in vitro experiments performed to evaluate expression levels of the modified alphavirus vector constructs described in Examples 1 and 2 above, and to investigate any differential behavior thereof (e.g., replication and protein expression).

In these experiments, the functionality of the modified alphavirus designs described in FIGS. 2B, 3B, 4B, and 5B were evaluated using the following assays. In these experiments, modified alphavirus srRNA vectors (SINV Girdwood with heterologous AR86 nsP1, or nsP2, or nsP3, or nsP4) encoding hemagglutinin precursor (HA) of the influenza A virus H5N1 (H5N1 HA) were also evaluated using the following assays.

In vitro transcription: Self-replicating RNA (srRNA was prepared by in vitro transcription using a plasmid DNA template linearized by enzymatic digestion. In these examples, the DNA was either linearized with NotI, which cuts downstream of the T7 terminator, or linearized with SapI, which cuts at the end of the poly(A). Bacteriophage T7 polymerase was used for in vitro transcription with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). srRNA was purified using phenol/chloroform extraction, LiCl precipitation, or column purification (Monarch® RNA Cleanup Kit, NEB). RNA concentration was determined by absorbance at 260 nm (Nanodrop, Thermo Fisher Scientific).

Replication: srRNA was transformed by electroporation into BHK-21 or Vero cells (e.g. 4D-Nucleofector™, Lonza). At 17-20 h following transformation, the cells were fixed and permeabilized (eBioscience™ Foxp3/Transcription Factor Staining Buffer Set, Invitrogen) and stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

Protein expression. RNA was transformed by electroporation into BHK-21 or Vero cells (e.g. 4D-Nucleofector™, Lonza). At 17-20 h following transformation, the cells were fixed and permeabilized (eBioscience™ Foxp3/Transcription Factor Staining Buffer Set, Invitrogen) and stained using an APC-conjugated anti-HA mouse monoclonal antibody (2B7, Abcam; APC: allophycocyanin) to quantify the frequency of HA protein+ cells and the mean fluorescence intensity (MFI) of the HA protein in individual cells by fluorescence flow cytometry (FIG. 7).

Additional experiments. BHK-21 or Vero cells are pre-treated with a titrated curve of recombinant IFN prior to electroporation of RNA and impacts on replication and protein expression for each vector are measured using the above assays.

A non-functional srRNA vector fails to exhibit a signal after staining srRNA-transfected cells to detect GOI expression, where functional srRNA vectors produce detectible GOI expression. In this experiment, GOI expression is quantified by mean fluorescence intensity (MFI) of cells that stained positive using the APC-conjugated anti-HA mouse monoclonal antibody. FIG. 7 illustrates that the base srRNA vectors which demonstrated RNA replication (FIG. 6) also exhibited expression after insertion of a GOI. These functional, GOI-expressing srRNAs can be used as practical vectors to induce pharmacokinetic effects (e.g. to elicit an immune response in a host).

### Example 5

### In Vivo Evaluation of Modified Alphavirus Vectors

This Example describes the results of in vivo experiments performed to evaluate any differential immune responses following vaccination with the modified alphavirus vector constructs described in Examples 1 and 2 above (e.g., both unformulated and LNP formulated vectors).

In these experiments, the functionality of the modified alphavirus designs described in FIGS. 2B, 3B, 4B, and 5B are evaluated using the following assays.

Mice and injections. Female C57BL/6 or BALB/c mice are purchased from Charles River Labs or Jackson Laboratories. On day of dosing, between 0.1-10 μg of material is injected intramuscularly split into both quadricep muscles. Vectors are administered either unformulated in saline, or LNP-formulated. Animals are monitored for body weight and other general observations throughout the course of the study. For immunogenicity studies, animals are dosed on Day 0 and Day 21. Spleens were collected at Day 35, and serum was isolated at Days 0, 14, and 35. For protein expression studies, animals are dosed on Day 0, and bioluminescence is assessed on Days 1, 3, and 7. In vivo imaging of luciferase activity is done using an IVIS system at the indicated time points.

LNP formulation. Replicon RNA (e.g., self-replicating RNA) is formulated in lipid nanoparticles using a microfluidics mixer and analyzed for particle size, polydispersity using dynamic light scattering and encapsulation efficiency. Molar ratios of lipids used in formulating LNP particles is 30% C12-200, 46.5% Cholesterol, 2.5% PEG-2K and 16% DOPE.

ELISpot. To measure the magnitude of Influenza-specific T cell responses, IFNγ ELISpot analysis is performed using Mouse IFNγ ELISpot PLUS Kit (HRP) (MabTech) as per manufacturer's instructions. In brief, splenocytes are isolated and resuspended to a concentration of 5×10⁶ cells/mL in media containing peptides representing either CD4+ or CD8+ T cell epitopes to HA, PMA/ionomycin as a positive control, or DMSO as a mock stimulation.

Intracellular cytokine staining. Spleens are isolated according to the methods outlined for ELISpots, and 1×10⁶ cells are added to cells containing media in a total volume of 200 μL per well. Each well contains peptides representing either CD4+ or CD8+ T cell epitopes to HA, PMA/ionomycin as a positive control, or DMSO as a mock stimulation. After 1 hour, GolgiPlug™ protein transport inhibitor (BD Biosciences) is added to each well. Cells are incubated for another 5 hours. Following incubation, cells are surface stained for CD8+(53-6.7), CD4+(GK1.5), B220 (B238128), Gr-1 (RB6-8C5), CD16/32 (M93) using standard methods. Following surface staining, cells are fixed and stained for intracellular proteins as per standard methods for IFNγ (RPA-T8), IL-2 (JES6-5H4), and TNF (MP6-XT22). Cells are then subsequently analyzed on a flow cytometer and the acquired FCS files analyzed using FlowJo software version 10.4.1.

Antibodies. Antibody responses to measure total HA-specific IgG are measured using ELISA kits from Alpha Diagnostic International as per manufacturer's instructions.

### Example 6

In Vitro Evaluation of Modified srRNA Vectors with Heterologous Nonstructural Protein Genes

This Example describes the results of in vitro experiments performed to evaluate expression levels of synthetic self-replicating RNAs (srRNAs) with a heterologous nonstructural protein and to investigate any differential behavior thereof (e.g., replication and protein expression).

In these experiments, synthetic srRNAs derived from the SINV strain Girdwood and AR86 were designed and subsequently evaluated, including control VEEV srRNAs with irrelevant transgenes (RBI296, RBI298), VEEV srRNAs encoding both IL-1RA and IL-12 in two configurations (RBI299, RBI300), SINV AR86-Girdwood Hybrid 1 srRNAs encoding both IL-1RA and IL-12 in two configurations (RBI307, RBI308), and SINV Girdwood srRNAs encoding both IL-1RA and IL-12 in two configurations (RBI309, RBI310).

In vitro transcription: srRNA was prepared by in vitro transcription from a SapI-linearized plasmid template with bacteriophage T7 polymerase by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). srRNA was then purified by LiCi precipitation. srRNA concentration was determined by absorbance at 260 nm (Nanodrop, Thermo Fisher Scientific).

Protein expression: srRNA was transformed by electroporation into BHK-21 cells (4D-Nucleofector™, Lonza). At 24 and 48 hours following transformation, conditioned media was collected from the cells. Secreted IL-1RA was evaluated in a bioactivity assay by pre-incubating HEK-Blue™ IL-1R cells (InvivoGen) with a range of concentrations of recombinant IL-1RA (Peprotech) or conditioned media. Recombinant IL-1B (Invivogen) was added to the cells and incubated overnight then the SEAP reporter was quantified using QUANTI-Blue™ (Invivogen) (FIG. 8A).

Secreted IL-12 was evaluated in a bioactivity assay by incubating a range of concentrations of recombinant IL-12 (Peprotech) or conditioned media on IL-12 bioassay cells (Promega) overnight in DMEM then the luciferase reporter was quantified using Bio-Glo™ Luciferase (Promega) (FIG. 8B).

### Example 8

In Vivo Evaluation of Modified srRNA Vectors with Heterologous Nonstructural Protein Genes

This Example describes the results of in vivo experiments performed to evaluate any differential immune responses following vaccination with self-replicating RNAs (srRNAs) with a heterologous nonstructural protein, as both unformulated and LNP formulated vectors.

In these experiments, synthetic srRNA constructs derived from SINV Girdwood-AR86 Hybrid 1 were designed and subsequently evaluated.

Mice and injections. Female C57BL/6 or BALB/c mice were purchased from Charles River Labs or Jackson Laboratories. On day of dosing, between 0.1-10 μg of material was injected intramuscularly split into both quadricep muscles. Vectors were administered either unformulated in saline, or LNP-formulated. Animals were monitored for body weight and other general observations throughout the course of the study. For immunogenicity studies, animals were dosed on Day 0 and Day 21. Spleens were collected at Day 14 and/or 35, and serum was isolated at Days 14, and/or 35.

LNP formulation. For some studies, srRNA was formulated in lipid nanoparticles (LNPs) using a microfluidics mixer and analyzed for particle size, polydispersity using dynamic light scattering and encapsulation efficiency. LNP are composed of an ionizable lipid, cholesterol, PEG-2K, and DOPE.

ELISpot. To measure the magnitude of antigen-specific T cell responses, IFNγ ELISpot analysis was performed using Mouse IFNγ ELISpot PLUS Kit (HRP) (MabTech) as per manufacturer's instructions. In brief, splenocytes are isolated and resuspended to a concentration of 2-5×10⁶ cells/mL in media containing peptides representing either peptide pools corresponding to rabies virus glycoprotein G, ESR1, HER2, or HER3, PMA/ionomycin as a positive control, or DMSO as a mock stimulation.

Antibodies. Neutralizing antibody responses to rabies virus are measured using rapid fluorescent focus inhibition test. In brief, serum dilutions are mixed with a standard amount of live rabies virus and incubated. If neutralizing anti-rabies antibodies are present, they will neutralize the virus. Next, cultured cells are added and the serum/virus/cells are incubated together. Uncoated rabies virus (i.e. that has not been neutralized by antibodies), will infect the cells and this can be visualized by microscopy. Calculation of the endpoint titer is made from the percent of virus infected cells observed on the slide.

In vivo immunogenicity of srRNAs encoding a viral antigen, rabies virus glycoprotein G, was assessed by evaluating antigen-specific splenic T cell responses by ELISpot (FIG. 9A) and anti-rabies neutralizing antibody titers from sera (FIG. 9B) after two immunizations. All srRNA-immunized groups showed robust T cell responses compared to saline controls (FIG. 9A), but differential responses were observed between srRNA vaccines. Similarly, all srRNA-immunized groups showed protective neutralizing antibody titers with some variations between srRNA vaccines (FIG. 9B). In addition to an infectious disease antigen, immunogenicity of srRNA-based vaccines to cancer antigens was assessed (FIG. 10). Each srRNA vaccine co-encoded sequences from ESR1, HER2, and HER3. Splenic T cell responses to these three antigens were determined using ELISpot analysis in mice having received two immunizations. Robust T cell responses were observed to all three targets, while the pattern of responses differed between srRNA vectors (FIG. 10).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A nucleic acid construct encoding a modified genome or RNA replicon of an alphavirus species, wherein at least one nonstructural protein (nsP), or a portion thereof, of the modified alphavirus genome or RNA replicon is heterologous relative to the remainder of the modified alphavirus genome or RNA replicon.

2. The nucleic acid construct of claim 1, wherein the at least one heterologous nsP or portion thereof is nsP1, nsP2, nsP3, nsP4, or a portion of any thereof, or a combination of any of the foregoing.

3. The nucleic acid construct of any one of claims 1 or 2, wherein the at least one heterologous nsP or portion thereof is derived from another strain of the same alphavirus species.

4. The nucleic acid construct of any one of claims 1 or 2, wherein the at least one heterologous nsP or portion thereof is derived from another alphavirus species.

5. The nucleic acid construct of any one of claims 1 to 4, wherein the modified alphavirus genome or RNA replicon is devoid of at least a portion of the nucleic acid sequence encoding one or more viral structural proteins.

6. The nucleic acid construct of any one of claims 1 to 5, wherein the modified viral genome or RNA replicon is devoid of a substantial portion of the nucleic acid sequence encoding one or more viral structural proteins.

7. The nucleic acid construct of any one of claims 1 to 6, wherein the modified viral genome or RNA replicon comprises no nucleic acid sequence encoding viral structural proteins.

8. The nucleic acid construct of any one of claims 1-7, further comprising one or more expression cassettes, wherein each of the expression cassettes comprises a promoter operably linked to a heterologous nucleic acid sequence.

9. The nucleic acid construct of claim 8, wherein at least one of the expression cassettes comprises a subgenomic (sg) promoter operably linked to a heterologous nucleic acid sequence.

10. The nucleic acid construct of claim 9, wherein the sg promoter is a 26S subgenomic promoter.

11. The nucleic acid construct of any one of claims 1-10, further comprising one or more untranslated regions (UTRs).

12. The nucleic acid construct of claim 11, wherein at least one of the UTRs is a heterologous UTR.

13. The nucleic acid construct of any one of claims 8-12, wherein at least one of expression cassettes comprises a coding sequence for a gene of interest (GOI).

14. The nucleic acid construct of claim 13, wherein the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide.

15. The nucleic acid construct of any one of claims 13-14, wherein the GOI encodes a polypeptide selected from the group consisting of an antibody, an antigen, an immune modulator, an enzyme, a signaling protein, and a cytokine.

16. The nucleic acid construct of any one of claims 13-15, wherein the coding sequence of the GOI is optimized for expression at a level higher than the expression level of a reference coding sequence.

17. The nucleic acid construct of any one of claims 13-16, wherein the coding sequence of the GOI is optimized for enhanced RNA stability.

18. The nucleic acid construct of any one of claims 1-17, wherein the alphavirus species is selected from the group consisting of Aura virus (AURAV), Babanki virus (BABV), Barmah Forest virus (BFV), Bebaru virus (BEBV), Buggy Creek virus, Caaingua virus, Cabassou virus, Chikungunya virus (CHIKV), Eastern equine encephalitis virus (EEEV), Eilat virus, Everglades virus (EVEV), Fort Morgan virus (FMV), Getah virus (GETV), Highlands J virus (HJV), Kyzylagach virus (KYZV), Madariaga virus (MADV), Mayaro virus (MAYV), Middelburg virus (MIDV), Mosso das Pedras virus, Mucambo virus (MUCV), Ndumu virus (NDUV), O‘nyong’nyong virus (ONNV), Pixuna virus (PIXV), Rio Negro virus (RNV), Ross River virus (RRV), Salmon pancreas disease virus (SPDV), Semliki Forest virus (SFV), Sindbis virus (SINV), Sleeping disease virus (SDV), Southern elephant seal virus (SESV), Tai Forest virus (TFV), Tonate virus, Trocara virus, Una virus (UNAV), Venezuelan equine encephalitis virus (VEEV), Western equine encephalitis virus (WEEV), and Whataroa virus (WHAV).

19. The nucleic acid construct of any one of claims 1-18, wherein the modified genome or RNA replicon is of a Sindbis virus (SINV).

20. The nucleic acid construct of claim 19, wherein the modified genome or RNA replicon is of a SINV strain Girdwood.

21. The nucleic acid construct of claim 20, wherein the at least one heterologous nsP or portion thereof of the modified genome or RNA replicon is derived from a SINV strain AR86.

22. The nucleic acid construct of any one of claims 20-21, wherein the at least one heterologous nsP or portion thereof is nsP1, nsP3, nsP4, or a portion of any thereof, or a combination of any of the foregoing.

23. The nucleic acid construct of claim 19, wherein the modified genome or RNA replicon is of a SINV strain AR86.

24. The nucleic acid construct of claim 23, wherein the at least one heterologous nsP or portion thereof of the modified SINV-AR86 genome or RNA replicon is derived from a SINV strain Girdwood.

25. The nucleic acid construct of any one of claims 23-24, wherein the at least one heterologous nsP or portion thereof of the modified SINV-AR86 genome or RNA replicon is derived from nsP2 of a SINV strain Girdwood.

26. The nucleic acid construct of any one of claims 1-25, wherein the nucleic acid construct is incorporated into a vector.

27. The nucleic acid construct of claim 26, wherein the vector is a self-replicating RNA (srRNA) vector.

28. The nucleic acid construct of any one of claims 1-27, wherein the nucleic acid construct comprising a nucleic acid sequence having at least 80%, at least 85%, at least 90%, at least 95%, at least 96%, at least 97%, at least 98%, at least 99%, or 100% sequence identity to a nucleic acid sequence selected from the group consisting of SEQ ID NOS: 1-4.

29. A recombinant cell comprising a nucleic acid construct according to any one of claims 1-28.

30. The recombinant cell of claim 29, wherein the recombinant cell is a eukaryotic cell.

31. The recombinant cell of claim 30, wherein the recombinant cell is an animal cell.

32. The recombinant cell of claim 31, wherein the animal cell is a vertebrate animal cell or an invertebrate animal cell.

33. The recombinant cell of claim 31, wherein the animal cell is an insect cell.

34. The recombinant cell of claim 33, wherein the insect cell is a mosquito cell.

35. The recombinant cell of claim 32, wherein the recombinant cell is a mammalian cell.

36. The recombinant cell of claim 32, wherein the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), canine kidney cell (MDCK), buffalo rat liver cell (BRL 3A), human lung cell (W138), human liver cell (Hep G2), mouse mammary tumor (MMT 060562), TRI cell, FS4 cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

37. A cell culture comprising at least one recombinant cell according to any one of claims 29-36, and a culture medium.

38. A method for functionalizing/engineering an alphavirus genome or RNA replicon, comprising:
(a) providing a non-functional alphavirus genome or RNA replicon;
(b) replacing a nonstructural protein (nsP), or a portion thereof, of the non-functional alphavirus genome or RNA replicon with a heterologous coding sequence for the corresponding nsP or portion thereof derived from a different alphavirus strain to generate a modified alphavirus genome or RNA replicon;
(c) assessing functionality of the modified alphavirus genome or RNA replicon;
(d) identifying the modified alphavirus genome or RNA replicon as being functional if the modified alphavirus genome or RNA replicon is capable of RNA replication and/or expression.

39. The method of claim 38, wherein the heterologous nsP or portion thereof is derived from another strain of the same alphavirus species.

40. The method of claim 38, wherein the heterologous nsP or portion thereof is derived from another alphavirus species.

41. The method of any one of claims 38-40, wherein the heterologous nsP or portion thereof is nsP1, nsP2, nsP3, nsP4, or a portion of any thereof.

42. The method of any one of claims 38-41, wherein the non-functionality of the alphavirus genome or RNA replicon is determined by a deficiency in self-replication within a host cell.

43. The method of any one of claims 38-42, the assessing functionality of the modified alphavirus genome or RNA replicon comprises an assay selected from the group consisting of: detection of RNA replication, detection of viral protein expression, detection of cytopathic effect (CPE), and detection of heterologous transgene expression.

44. A transgenic animal comprising a nucleic acid construct according to any one of claims 1-28.

45. The transgenic animal of claim 44, wherein the animal is a vertebrate animal or an invertebrate animal.

46. The transgenic animal of claim 45, wherein the animal is an insect.

47. The transgenic animal of claim 45, wherein the animal is a mammal.

48. The transgenic animal of claim 46, wherein the mammal is a non-human mammal.

49. A method for producing a polypeptide of interest, comprising (i) rearing a transgenic animal according to any one of claims 44-48, or (ii) culturing a recombinant cell comprising a nucleic acid construct according to any one of claims 1-28 under conditions wherein the recombinant cell produces the polypeptide encoded by the GOI.

50. A method for producing a polypeptide of interest in a subject, comprising administering to the subject a nucleic acid construct according to any one of claims 1-28.

51. The method of any one of claims 49-50, wherein the subject is vertebrate animal or an invertebrate animal.

52. The method of claims 49-51, wherein the animal is an insect.

53. The method of any one of claims 49-51, wherein the subject is a mammalian subject.

54. The method of claim 53, wherein the mammalian subject is a human subject.

55. A recombinant polypeptide produced by the method of any one of claims 49-54.

56. A pharmaceutical composition comprising a pharmaceutically acceptable excipient and:
(a) a nucleic acid construct of any one of claims 1-28;
(b) a recombinant cell of any one of claims 29-36; and/or
(c) a recombinant polypeptide of claim 55.

57. The pharmaceutical composition of claim 56, comprising a nucleic acid construct of any one of claims 1-28, and a pharmaceutically acceptable excipient.

58. The pharmaceutical composition of claim 56, comprising a recombinant cell of any one of claims 29-36, and a pharmaceutically acceptable excipient.

59. The pharmaceutical composition of claim 56, comprising a recombinant polypeptide of claim 55, and a pharmaceutically acceptable excipient.

60. The pharmaceutical composition of any one of claims 56-59, wherein the composition is formulated in a liposome, a lipid-based nanoparticle (LNP), or a polymer nanoparticle.

61. The pharmaceutical composition of any one of claims 56-60, wherein the composition is an immunogenic composition.

62. The pharmaceutical composition of claim 61, wherein the immunogenic composition is formulated as a vaccine.

63. The pharmaceutical composition of any one of claims 56-60, wherein the composition is substantially non-immunogenic to a subject.

64. The pharmaceutical composition of any one of claims 56-63, wherein the pharmaceutical composition is formulated as an adjuvant.

65. The pharmaceutical composition of any one of claims 56-64, wherein the pharmaceutical composition is formulated for one or more of intranasal administration, transdermal administration, intraperitoneal administration, intramuscular administration, intranodal administration, intratumoral administration, intraarticular administration, intravenous administration, subcutaneous administration, intravaginal administration, and oral administration.

66. A method for inducing a pharmacodynamic effect in a subject in need thereof, the method comprises administering to the subject a composition comprising:
(a) a nucleic acid construct of any one of claims 1-28;
(b) a recombinant cell of any one of claims 29-36;
(c) a recombinant polypeptide of claim 55; and/or
(d) a pharmaceutical composition of any one of claims 56-65.

67. The method of claim 66, wherein the pharmacodynamic effect comprises eliciting an immune response in the subject.

68. A method for preventing and/or treating a health condition in a subject in need thereof, the method comprises prophylactically or therapeutically administering to the subject a composition comprising:
(a) a nucleic acid construct of any one of claims 1-28;
(b) a recombinant cell of any one of claims 29-36;
(c) a recombinant polypeptide of claim 55; and/or
(d) a pharmaceutical composition of any one of claims 56-64.

69. The method of any one of claims 66-67, wherein the condition is a proliferative disorder or a microbial infection.

70. The method of any one of claims 66-69, wherein the subject has or is suspected of having a condition associated with proliferative disorder or a microbial infection.

71. The method of any one of claims 66-70, wherein the administered composition results in an increased production of interferon in the subject.

72. The method of any one of claims 66-71, wherein the composition is administered to the subject individually as a single therapy (monotherapy) or as a first therapy in combination with at least one additional therapies.

73. The method of claim 72, wherein the at least one additional therapies is selected from the group consisting of chemotherapy, radiotherapy, immunotherapy, hormonal therapy, toxin therapy, targeted therapy, and surgery.

74. A kit for inducing a pharmacodynamic effect, for eliciting an immune response, for the prevention, and/or for the treatment of a health condition or a microbial infection, the kit comprising:
(a) a nucleic acid construct of any one of claims 1-28;
(b) a recombinant cell of any one of claims 29-36;
(c) a recombinant polypeptide of claim 55; and/or
(d) a pharmaceutical composition of any one of claims 56-65.
